SEQUENCE LISTING (4 sequences)

SEQ ID NO: 1 - DNA, 2870 bp, Lactobacillus rhamnosus
ctgtaatggtgacgtaacattgacacagtaaagtagcctttagttaattaattaagggtgaggtcaaacatgaaattcaataaagcaatgatgacattggttgcagcagttaccttagcgggttctgttagcgccgtaacaccggttttcgctgacacaagtgccagcatcgcatctaacaagagcgaaaccaacgatttattaaagcaaatcgaagcagctaacactgaagtgatcaacctcaacaagcagattgatgcaaagaatggcgaaatcagtgacgccactgccaagatcagtgcaacggatgccaagatcgcatcgttgagtggcgaaatcaccgctgctcaaaagaacgtcgcagcccggaagaacaacttgaaggatcaattgatttcccttcaaaagaaagccggcagttcagttagcggcaatgtctatattgatttcgtgttgaactcacaaagtctatctgacttgattgcccgtaccatgacagtcggcaagttaagtcaagccagcaaagacgctttggatgcggtaaccgtcgccaaagataagttagccgctttgaagagtgaacaggaaacggcacgtcagaccttggtttcgactaaagcatccctcgaaacgcaaaagtcacagctagagacccttcaaaagaccgcaagcgataagcaagatgctttgaacaaagaaattgccgatcacaaagacgaattggttgcactccaaagtcaatttgctcaagaacaatcagaagcagccaaagcaacccaggctgccttgaagacagcggctgcatcaactgcttcaagttctaccagctcaacttcaaataagagtgccaacagcagtgttctttcgactggtacctcatcaaccaacacttcttccaacagcggtgcctcatctactgtgatcagcagcaacactgcttcaggtagtggcagccatgctgattacagcggttcaggtaacacttatccttggggtcagtgcacatggtacgttaagtctgttgcttcatgggcaggcaatggctggggcaacggtgctgaatggggcgcttccgctgcagcagccggcttcacggttaaccacaccccggcagcaggttccatcatcgtcttcgctgctggtcaatctgttggcggccaatggacagctgatggctcttatggtcacgttgcttatgttcaatccgtttctggcgacagcgttacgatcactcaaggcggtatgggcttcagctcaccaaccggtcctaacacccagaccatctctggtgccagcagctacgtttacatccaccggtaatttttacccacccttaatatcacgtcttgggacctcaccaatgaatcccaacctaaaaataccgttgaaataaataaccctctgaacaactgtcatttgaagacatgttcagagggttattttttatgtgtattgacgcaaaagtaacttaatggaagtgggcgttgccattttactgacttaagatgaaagcttcctcctctaaatgcccgctccctcagcagtcacaagcaacatcacaagttgcacaatcatcaagatagaaactattggcgcggctgattttaacaaagtccccgatgtcccattgttcccgtttagcgatctcattaacgatcactccgaaacgctggcggaatttgtaaatgtaacagaaaatcgcatcctcgtggcgtacatcaggcccggcaacaaatacatttttcgcctgagttgattcatcaacgtcattcaaagcaatttcgccatcattattttcaaataattgtggccagagttttaacgggccggcatcaaaacctgtgcatagaacgggcttatacggcgtcgtaatcgtacgtccgtctgtaaaccacaattgatactgggaatcctgggtgcgcgtgacggtcttcaacgcaatcttctcttgtaacgtgattctgttagcaacttccgctggcaaatccaagaatcgccgccgtgtgtacggtgccaaccgaatacttgggtcggccgatgccgcattcagacccgtttccttggtcaacaaaagtaccttatggcctagcgttgccaaattgatagccgcatcaactccactttcgtttccgccaatcacaatctgggttttccccgtcaagtcctgccacgaggtcacttgctgatagtgaatgacgttttccggatgatcggttaatttagggaaagcatactgaccgactgccatgatcaagtacttggtggtcacgacgccgtgattcgtttcgagttgataaccttcagtagtcggtgaaattgcaccaacgcgcgtatgttcagcgatcggtaatgcgtaagtatccgccaacaatttaagataatcggcaaattgtgaaccactcagatgctctgcaccaagcgtatacgcaggcgaggtctccatactgacggcattcaaatccggcatgccgaatccgttactggtaaaggatggcgtaatgaatcgcgtttcttttggccaacgtcggaaacttgcgccaatttcttcctgctcaagaatctgaaattgactgcagccaagttgttctaacatgataccgataccaacgcccgctgctccaccaccaataatcgtcatttgattcatccaaatcgctccttttgtcaattcaaccgttcactgtcaagattcatcacccgtgttacgccagcttcacacgccttagcaactaactgcgcaaccattgtgccaataaccgtcaacccagccgcgaccaccacagctaaaagttcaaccactgcgaacttccccgcggtcgctattgcatgagtcccattaaagaattggacaatcattaagccaacagccc

SEQ ID NO: 2 - DNA, 1251 bp, Lactobacillus rhamnosus
atgaaattcaataaagcaatgatgacattggttgcagcagttaccttagcgggttctgttagcgccgtaacaccggttttcgctgacacaagtgccagcatcgcatctaacaagagcgaaaccaacgatttattaaagcaaatcgaagcagctaacactgaagtgatcaacctcaacaagcagattgatgcaaagaatggcgaaatcagtgacgccactgccaagatcagtgcaacggatgccaagatcgcatcgttgagtggcgaaatcaccgctgctcaaaagaacgtcgcagcccggaagaacaacttgaaggatcaattgatttcccttcaaaagaaagccggcagttcagttagcggcaatgtctatattgatttcgtgttgaactcacaaagtctatctgacttgattgcccgtaccatgacagtcggcaagttaagtcaagccagcaaagacgctttggatgcggtaaccgtcgccaaagataagttagccgctttgaagagtgaacaggaaacggcacgtcagaccttggtttcgactaaagcatccctcgaaacgcaaaagtcacagctagagacccttcaaaagaccgcaagcgataagcaagatgctttgaacaaagaaattgccgatcacaaagacgaattggttgcactccaaagtcaatttgctcaagaacaatcagaagcagccaaagcaacccaggctgccttgaagacagcggctgcatcaactgcttcaagttctaccagctcaacttcaaataagagtgccaacagcagtgttctttcgactggtacctcatcaaccaacacttcttccaacagcggtgcctcatctactgtgatcagcagcaacactgcttcaggtagtggcagccatgctgattacagcggttcaggtaacacttatccttggggtcagtgcacatggtacgttaagtctgttgcttcatgggcaggcaatggctggggcaacggtgctgaatggggcgcttccgctgcagcagccggcttcacggttaaccacaccccggcagcaggttccatcatcgtcttcgctgctggtcaatctgttggcggccaatggacagctgatggctcttatggtcacgttgcttatgttcaatccgtttctggcgacagcgttacgatcactcaaggcggtatgggcttcagctcaccaaccggtcctaacacccagaccatctctggtgccagcagctacgtttacatccaccggtaa

SEQ ID NO: 3 - protein, 416 aa, Lactobacillus rhamnosus
MKFNKAMMTLVAAVTLAGSVSAVTPVFADTSASIASNKSETNDLLKQIEAANTEVINLNKQIDAKNGEISDATAKISATDAKIASLSGEITAAQKNVAARKNNLKDQLISLQKKAGSSVSGNVYIDFVLNSQSLSDLIARTMTVGKLSQASKDALDAVTVAKDKLAALKSEQETARQTLVSTKASLETQKSQLETLQKTASDKQDALNKEIADHKDELVALQSQFAQEQSEAAKATQAALKTAAASTASSSTSSTSNKSANSSVLSTGTSSTNTSSNSGASSTVISSNTASGSGSHADYSGSGNTYPWGQCTWYVKSVASWAGNGWGNGAEWGASAAAAGFTVNHTPAAGSIIVFAAGQSVGGQWTADGSYGHVAYVQSVSGDSVTITQGGMGFSSPTGPNTQTISGASSYVYIHR

SEQ ID NO: 4 - protein, 387 aa, Lactobacillus rhamnosus
TSASIASNKSETNDLLKQIEAANTEVINLNKQIDAKNGEISDATAKISATDAKIASLSGEITAAQKNVAARKNNLKDQLISLQKKAGSSVSGNVYIDFVLNSQSLSDLIARTMTVGKLSQASKDALDAVTVAKDKLAALKSEQETARQTLVSTKASLETQKSQLETLQKTASDKQDALNKEIADHKDELVALQSQFAQEQSEAAKATQAALKTAAASTASSSTSSTSNKSANSSVLSTGTSSTNTSSNSGASSTVISSNTASGSGSHADYSGSGNTYPWGQCTWYVKSVASWAGNGWGNGAEWGASAAAAGFTVNHTPAAGSIIVFAAGQSVGGQWTADGSYGHVAYVQSVSGDSVTITQGGMGFSSPTGPNTQTISGASSYVYIHR

(19) United States
(12) Patent Application Publication
(10) Pub. No.: US 2018/0333442 A1
(43) Pub. Date: Nov. 22, 2018

(54) PROBIOTIC COMPOSITIONS AND METHODS OF USE THEREOF

(71) Applicant: The Regents of the University of Michigan, Ann Arbor, MI (US)

(72) Inventor: Chung Owyang, Ann Arbor, MI (US)

(21) Appl. No.: 15/983,884

(22) Filed: May 18, 2018

Related U.S. Application Data
(60) Provisional application No. 62/508,127, filed on May 18, 2017.

(51) Int. Cl. A61K 35/747; A61K 38/16; C07K 14/32

(52) U.S. Cl. CPC A61K 35/747; C07K 14/32; A61K 38/164

(57) ABSTRACT

Methods and compositions are provided for treating (e.g., therapeutically or prophylactically) gastrointestinal diseases, disorders, and conditions. In particular, one aspect provides compositions comprising Lactobacillus (e.g., Lactobacillus rhamnosus GG or other Lactobacillus species) p40 protein (e.g., isolated, recombinant, or overexpressed p40 protein) and methods of using same for treating a gastrointestinal disease, disorder, or condition.

This application claims priority to U.S. Provisional Patent Application No. 62/508,127 filed May 18, 2017, incorporated herein by reference in its entirety.

### FIELD OF THE INVENTION

Methods and compositions are provided for treating (e.g., therapeutically or prophylactically) gastrointestinal diseases, disorders, and conditions. In particular, one aspect provides compositions comprising Lactobacillus (e.g., Lactobacillus rhamnosus GG or other Lactobacillus species) p40 protein (e.g., isolated, recombinant, or overexpressed p40 protein) and methods of using same for treating a gastrointestinal disease, disorder, or condition. Another aspect provides methods and compositions for treating a subject with a gastrointestinal disease, disorder, or condition by administering to the subject a composition that includes isolated, recombinant, or overexpressed Lactobacillus p40 protein. Yet another aspect of the invention includes pharmaceutical compositions (e.g., for altering microbiota in a subject or for treating a gastrointestinal disease, disorder, or condition in a subject) comprising a therapeutically effective amount of isolated, recombinant, or overexpressed Lactobacillus p40 protein. Compositions and methods of the invention find use in both clinical and research settings, for example, within the fields of biology, immunology, medicine, and gastroenterology.

### BACKGROUND

The characterization of the human microbiome in various disease states suggests that the human microbial environment plays a critical role in both the maintenance of health and the pathogenesis of disease. As the most densely populated and diverse of the microbial communities, the intestinal microbiome may be particularly important. The intestinal microbiome is a complex system, providing an environment or niche for a community of many different species or organisms including diverse strains of bacteria. Hundreds of different species may form a commensal community in the gastrointestinal tract in a healthy person and this complement of organisms evolves from the time of birth to ultimately form a functionally mature microbial population by about 3 years of age. Interactions between microbial strains in these populations and between microbes and the host (e.g. the host immune system) shape the community structure, with availability of and competition for resources affecting the distribution of microbes. Diet is involved in shaping the GI tract flora.

The microbiome/microbiota provides the host with significant benefits that include resistance to colonization by a broad spectrum of pathogens, nutrient biosynthesis and absorption, and immune system stimulation that maintains a healthy gut epithelium and an appropriately controlled systemic immunity.

Disrupted symbiosis (“dysbiosis”) refers to physiologic states where microbiota functions are lost or altered resulting in increased susceptibility to pathogens, altered metabolic profiles, and/or induction of proinflammatory conditions resulting in local or systemic inflammation or autoimmunity

Accordingly, the intestinal microbiota plays a significant role in the pathogenesis of many diseases and disorders. Many of these diseases and disorders are chronic conditions that significantly decrease a subject's quality of life and can be ultimately fatal. For example, disruption of intestinal epithelial barrier function appears to play an important role in the pathogenesis of chronic inflammatory intestinal disorders. Altered gut permeability may increase bacterial load and dietary antigens in the lamina propia leading to mucosal inflammation.

### SUMMARY OF THE INVENTION

Methods and compositions are provided for treating (e.g., therapeutically or prophylactically) gastrointestinal diseases, disorders, and conditions. In particular, one aspect provides compositions comprising Lactobacillus (e.g., Lactobacillus rhamnosus or other Lactobacillus species) p40 protein (e.g., isolated, recombinant, or overexpressed p40 protein) and methods of using same for treating a gastrointestinal disease, disorder, or condition. Another aspect provides methods and compositions for treating a subject with a gastrointestinal disease, disorder, or condition by administering to the subject a composition that includes isolated, recombinant, or overexpressed Lactobacillus p40 protein. Yet another aspect of the invention includes pharmaceutical compositions (e.g., for altering microbiota in a subject or for treating a gastrointestinal disease, disorder, or condition in a subject) comprising a therapeutically effective amount of isolated, recombinant, or overexpressed Lactobacillus p40 protein. Compositions and methods of the invention find use in both clinical and research settings, for example, within the fields of biology, immunology, medicine, and gastroenterology.

Accordingly, in one embodiment, the invention provides compositions comprising Lactobacillus (e.g., Lactobacillus rhamnosus (e.g., Lactobacillus rhamnosus GG) or other Lactobacillus species) p40 protein for treating a gastrointestinal disease, disorder, or condition in a subject. In one embodiment, the composition is a cell based composition comprising bacterial cells modified (e.g., genetically engineered) to express (e.g., overexpress) or to harbor Lactobacillus p40. In another embodiment, the composition is a cell based composition comprising bacterial cells administered with isolated (e.g., recombinant) Lactobacillus p40. In still another embodiment, the composition is a cell based composition comprising bacterial cells and further including one or more bacterial cell line(s) modified (e.g., genetically) to express (e.g., overexpress) Lactobacillus p40. In one embodiment, a cell based composition comprises purified microbiota from phyla such as Bacteroidetes, Proteobacteria, Firmicutes and Verrucomicrobia or orders such as Bacteroidales, Enterobacteriales, Clostridiales, and Verrucomicrobiales, or genera such as Alistipes, Escherichia, Clostridium, Lactobacillus or Akkermansia (e.g., the microbiota are modified to overexpress Lactobacillus p40, or the microbiota are combined with isolated, purified, and/or recombinant p40, or the microbiota are combined with Lactobacillus bacteria that have been genetically modified to overexpress p40). The invention is not limited by the specific species of Lactobacillus p40 used. Indeed, any Lactobacilli p40 may be used including, but not limited to, L. acidophilus, L. brevis, L. bulgaricus, L. casei, L. fermentum, L. gasseri, L. paracasei, L. plantarum, L. reuteri, L. rhamnosus, L. salivarius, and/or L. sporogenes. In a preferred embodiment, L. rhamnosus p40 is used. In one embodiment, p40 from two or more different species of Lactobacilli are used. P40 may be isolated and/or purified p40. In another embodiment, p40 is recombinant p40.

In another embodiment, one or more species of Lactobacilli are genetically modified to overexpress p40 (e.g., one or more species of Lactobacilli are genetically modified to overexpress p40 and are used independently and/or combined with other microbiota (e.g., that are not genetically modified) in a composition of the invention). In another embodiment, p40 is isolated and or purified from Lactobacilli (e.g., one or more species of Lactobacilli genetically modified to overexpress p40). In yet another embodiment, a cell lysate (e.g., prepared from one or more species of Lactobacilli genetically modified to overexpress p40) is used in a composition of the invention. In a further embodiment, the invention provides a composition (e.g., for use in a therapeutic method described herein) comprising a cell lysate harvested from bacterial cells (e.g., one or more strains of Lactobacilli genetically modified to overexpress p40) and also includes one or more bacterial cell species (e.g., one or more microbiota from phyla such as Bacteroidetes, Proteobacteria, Firmicutes and Verrucomicrobia or orders such as Bacteroidales, Enterobacteriales, Clostridiales, and Verrucomicrobiales, or genera such as Alistipes, Bacteroides, Escherichia, Clostridium, Lactobacillus or Prevotella, Parabacteroides and/or Ruminococcus). In another embodiment, the invention provides a composition (e.g., for use in a therapeutic method described herein) comprising a cell lysate (e.g., harvested from one or more species of Lactobacilli genetically modified to overexpress p40), one or more microbiota from phyla such as Bacteroidetes, Proteobacteria, Firmicutes and Verrucomicrobia or orders such as Bacteroidales, Enterobacteriales, Clostridiales, and Verrucomicrobiales, or genera such as Alistipes, Bacteroides, Escherichia, Clostridium, Lactobacillus or Prevotella, Parabacteroides and/or Ruminococcus, and includes isolated and/or recombinant p40.

In one embodiment, a composition comprising Lactobacillus (e.g., Lactobacillus rhamnosus or other Lactobacillus species) p40 protein (e.g., isolated, recombinant, or overexpressed p40 protein) is used in a method for treating (e.g., therapeutically and/or prophylactically) a gastrointestinal disease, disorder, or condition. For example, in one embodiment, the invention provides a method of treating a subject with a gastrointestinal disease, disorder, or condition by administering to the subject a therapeutically effective amount of a composition comprising isolated, recombinant, and/or overexpressed Lactobacillus p40 protein (e.g., one or more species of Lactobacilli genetically modified to overexpress p40 (e.g., used independently and/or combined with other microbiota (e.g., that are not genetically modified))). The invention is not limited by the type of gastrointestinal disease, disorder, or condition. In one embodiment, the disorder is irritable bowel syndrome (IBS). In another embodiment, the disease is inflammatory bowel disease. In still another embodiment, the condition is obesity. In another embodiment, the condition is fatty liver disease. In one embodiment, the subject is one with a disease, disorder, or condition that would benefit from maintaining and/or improving mucosal epithelial barrier function (e.g., via administration of a composition of the invention (e.g., a composition comprising isolated, recombinant, and/or overexpressed Lactobacillus p40 protein (e.g., one or more species of Lactobacilli genetically modified to overexpress p40 (e.g., used independently and/or combined with other microbiota (e.g., that are not genetically modified))))). In another embodiment, the subject is one with a disease, disorder, or condition that would benefit from the prevention or amelioration of intestinal mucosal tight junction damage (e.g., via administration of a composition of the invention (e.g., a composition comprising isolated, recombinant, and/or overexpressed Lactobacillus p40 protein (e.g., one or more species of Lactobacilli genetically modified to overexpress p40 (e.g., used independently and/or combined with other microbiota (e.g., that are not genetically modified))). Exemplary diseases, disorders, or conditions that may be treated using compositions and methods of the invention include, but are not limited to, metabolic syndrome, insulin deficiency, insulin-resistance related disorders, glucose intolerance, diabetes, non-alcoholic fatty liver, abnormal lipid metabolism, as well as other diseases, disorders, or conditions described herein.

The invention also provides methods for delivering a composition of the invention (e.g., one or more species of Lactobacilli genetically modified to overexpress p40 used independently and/or combined with other microbiota (e.g., that are not genetically modified), p40 isolated and/or purified from Lactobacilli genetically modified to overexpress p40, and/or cell lysate(s) from one or more species of Lactobacilli genetically modified to overexpress p40) to a subject (e.g., to a target location within the subject). For example, p40 can be recombinantly produced (e.g., in large quantities using genetic engineering techniques (e.g., those described herein and/or known in the art)) and delivered to patients. The invention is not limited by the type or route of administration. In some embodiments, a composition of the invention is delivered to a subject (e.g., a subject with inflammatory bowel disease (IBD), or a subject with irritable bowel syndrome (IBS)) using timed released capsules. In another embodiment, genetically engineered bacteria (e.g., one or more strains of lactobacilli modified to overexpress p40) are generated and are used for administration to a subject in the same way a conventional probiotic is used. In another embodiment, the composition is directly delivered to the stomach, the small intestine, and/or the large intestine of the subject. Compositions can also be formulated for oral delivery. Delivery methods can also include administering a composition of the invention to a subject and also performing a surgical procedure selected from gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, duodenal endoluminal barrier, similar manipulations of the gastrointestinal tract, and other gastrointestinal bariatric and metabolic procedures. Delivery methods may also include administering an additional agent, such as an antibiotic and/or an osmotic laxative, to the subject before, concurrent with, and/or after administration of the composition.

Compositions of the invention may be formulated as a pharmaceutical composition that includes a pharmaceutically acceptable carrier and administered alone or co-administered with one or more other pharmaceutical compositions. In other embodiments, a therapeutically effective amount of such compositions can be contained in food, drink, dietary supplement, and/or food additive to be consumed by a subject.

The invention is not limited by the type or form of p40 used. For example, in one embodiment, p40 expressed or harbored in a cell is free p40 (e.g., p40 polypeptide or oligomers of p40). Full length p40, or a biologically active fragment thereof (e.g., that prevents epithelial barrier damage, enhances junction protein synthesis, and/or enhances mucosa protection) may be used. For example, all or a portion of the nucleic acid sequence of SEQ ID NO: 1 or SEQ ID NO: 2 may be used (e.g., to express (e.g., using an expression vector to express in vivo or in vitro) p40). Any p40 peptide, or fragment thereof, that is biologically active (e.g., that prevents epithelial barrier damage, enhances junction protein synthesis, and/or enhances mucosa protection) finds use in the invention. For example, in some embodiments, p40 used in the compositions and methods of the invention comprises an amino acid sequence having at least 75%, 80%, 85%, 90%, 95%, 97%, 98%, 99% or more sequence identity to the amino acid sequence of SEQ ID NO: 3 (e.g., shown in FIG. 11A). In another embodiment, p40 used in the compositions and methods of the invention comprises an amino acid sequence having at least 75%, 80%, 85%, 90%, 95%, 97%, 98%, 99% or more sequence identity to the amino acid sequence of SEQ ID NO: 4 (e.g., shown in FIG. 11B), which is full length p40 protein sequence with the secreting signal, N-terminal 29 amino acids removed. The invention is not limited to any particular fragment of p40. Indeed, any fragment of p40 that is known or shown to be biologically active (e.g., that prevents epithelial barrier damage, enhances junction protein synthesis, and/or enhances mucosa protection) can be used. Furthermore, fragments of p40 can be assessed and identified as biologically active using methods described herein and/or methods known in the art. In some embodiments, full length p40, or a biologically active fragment thereof, is used (e.g., is co-administered) with a one or more other biologically active substances (e.g., to prevent epithelial barrier damage, to enhance junction protein synthesis, and/or to enhance mucosa protection) in a composition and/or method of the invention.

In one embodiment, administration of a composition of the invention and optionally one or more other therapeutic agents results in enhanced therapeutic efficacy and/or potency relative to administration of the composition of the invention or the one or more other therapeutic agents alone. The invention is not limited by the route or frequency of administration of a composition of the invention. Any suitable route of administration can be used to introduce a composition of the invention into a subject including, but not limited to, intravenous, subcutaneous administration, or other route or means described herein and/or known in the art. For example, local or systemic delivery can be accomplished by administration comprising administration of the combination into body cavities, by parenteral introduction comprising intramuscular, intravenous, intraportal, intrahepatic, peritoneal, subcutaneous, and/or intradermal administration. A composition of the invention may be administered proceeding, following, or in lieu of other treatments and/or therapies for treating and/or preventing IBD, IBS, or other gastroenteral disease, disorder and/or condition. In one embodiment, following administration, a response (e.g., epithelial barrier leakage and/or permeability) is detected wherein the response is not detected in a subject prior to administering the composition. In another embodiment, subsequent to detecting one or more responses in a subject, treatment of the patient is modified based on the status of the response(s) detected in the patient.

The invention also provides methods of manufacturing any one of the compositions comprising p40 of the invention, or a combination thereof, described herein.

The invention further provides methods of using one or more of the compositions of the invention for treating (e.g., therapeutically and/or prophylactically) a gastrointestinal disease, disorder, or condition in a subject (e.g., an IBD patient, and IBS patient, or other subject described herein). In a further embodiment, following administration of a composition of the invention to a subject/patient, one or more physiologic responses in the subject is detected (e.g., wherein the one or more physiologic responses are not detected the patient prior to administering the composition). In one embodiment, because the amount of p40 administered to a patient can be controlled and/or monitored (e.g., due to the exact amount of recombinant p40 present in a composition of the invention being known and/or knowable), the invention also provides methods of tailoring the amount of p40 administered to a subject (e.g., so as to provide customized care based upon a subject's disease, disorder, and/or condition).

### DESCRIPTION OF THE DRAWINGS

FIGS. 1A-B show gene expression levels of occludin and ZO-1 (FIGS. 1A and 1B, respectively) in mouse enteroids and that metabolites of Lactobacillus rhamnosus GG (LGG) prevent IFN-gamma-induced downregulation of tight junction proteins in mouse enteroids.

FIG. 2 shows Colloidal Blue Staining of recombinantly produced p40.

FIG. 3 shows Western blots indicating the binding of polyclonal anti-p40 antibodies against recombinant p40 and LGG supernatant and the absence of binding after immunodepletion of p40 from LGG supernatant.

FIG. 4 shows the absence of cleaved caspase-3 (green fluorescence) in p40 treated human enteroids indicating that p40 improves mucosal permeability in the absence of immune modulation and apoptosis.

FIG. 5 shows real time PCR after incubation of human enteroids with INFγ for 24 hr and the resulting 30% and 20% downregulation of gene expression of occludin and ZO-1, respectively, as well as the ability of p40 to negate/eliminate the downregulation of both genes (P<0.05).

FIGS. 6A-B show immunofluorescence staining of human enteroids. Enteroids retained 50% of FD4 at 24 hr (control). Treatment of the enteroids with INF gamma impaired permeability resulting in 20% retention of the dye (IFN-γ). Administration of p40 prevented excessive leakage of dye evoked by INF gamma. * P<0.05 (IFN-γ+p40).

FIGS. 7A-B show that soluble p40 from LGG supernatant prevents downregulation of ZO-1 and occludin gene expression induced by IBS-FSN. Incubation of human colonoids with Fecal supernatant from IBS-D patients induced epithelial barrier damage resulting in a 40% and 50% reduction in gene expression of occludin and ZO-1 (IBS). Treatment with p40 prevented the reduction and normalized the gene expression of ZO-1 and occludin (p40+IBS).

FIG. 8 shows that p40 produced by lactobacillus is responsible for enhanced synthesis of tight junction proteins. Administration of IBS-D patients fecal supernatant to human colonoids reduced protein expression of ZO-1 and Occludin (IBS-FSN). Pretreatment of the human colonoids with p40 prevented the reduction of ZO-1 and Occludin expression evoked by fecal supernatant of IBS-D patients (IBS-FSN+LGG-S, and IBS-FSN+p40). Removal of p40 by immunodepletion abolished the protective effects of LGG supernatant (IBS-FSN+LGG-p40).

FIG. 9 shows that p40 prevents IBS-FSN induced disruption of mucosal permeability. Human colonoids retained 70% of FD4 over 10 h (control). Treatment of the colonoids with IBS-FSN impaired permeability resulting in 10% retention of the dye at 10 h (IBS-FSN). Administration of soluble protein p40 prevented leakage of dye evoked by IBS-FSN (IBS-FSN-+p40).

FIGS. 10A-B show Lactobacillus rhamnosus GG (LGG) p40 (A) DNA sequence and (B) mRNA sequence.

FIGS. 11A-B shows (A) full length Lactobacillus rhamnosus GG (LGG) p40 amino acid sequence, and (B) p40 amino acid sequence with the N-terminal 29 amino acid secretion signal removed.

FIG. 12 shows that intracolonic perfusion of p40 protein prevented colonic inflammation evoked by administration of IBS fecal supernatant. RT-PCR measurement of cytokines in rats following intracolonic infusion of fecal supernatant from IBS patients. mRNA levels represented as fold change in each target mRNA after normalization to GAPDH. Fecal supernatant induced increases in ILIβ, IL6, IFNγ and ILI0 gene expression indicating low grade mucosal inflammation (*P<0.05). These changes were prevented by pretreatment with intracolonic infusion of P40 protein (# P<0.05). P values determined by 2 tailed Student's tests *P<0.05 IBS fecal supernatant vs PBS; # P<0.05 P40+IBS fecal supernatant vs IBS fecal supernatant.

FIG. 13 shows intracolonic perfusion of p40 prevented epithelial dysfunction and reduced expression of junction proteins evoked by infusion of IBS fecal supernatant in rats. (Panel A) Intracolonic infusion of fecal supernatant from IBS patients increased gut permeability as measured by TEER and (Panel B) decreased Z01 and occludin (OCLN) gene expression. Pretreatment with colonic administration of P40 protein prevented changes in gut permeability and junction protein expression (n=4-6 per group) *P<0.05 IBS vs PBS; # P<0.05 P40+IBS fecal supernatant vs IBS fecal supernatant.

FIG. 14 shows IBS fecal supernatant-induced visceral hypersensitivity is prevented by p40 protein. Mean amplitudes of abdominal muscle contractions are expressed as an area under the curve (AUC) after baseline subtraction (n=4-6 per group) *P<0.05, IBS fecal supernatant vs PBS # P<0.05, IBS fecal supernatant+P40 vs IBS fecal supernatant.

### DEFINITIONS

To facilitate an understanding of the present invention, a number of terms and phrases are defined below:

“Microbiota” is used herein to refer to the community of microorganisms that occur (sustainably or transiently) in and on an animal subject, typically a mammal such as a human, including eukaryotes, archaea, bacteria, and viruses (including bacterial viruses i.e., phage).

“Microbiome” refers to the genetic content of the communities of microbes that live in and on the human body, both sustainably and transiently, including eukaryotes, archaea, bacteria, and viruses (including bacterial viruses (i.e., phage)), wherein “genetic content” includes genomic DNA, RNA such as ribosomal RNA, the epigenome, plasmids, and all other types of genetic information

“Intestinal Enteroid Culture,” “enteroid culture” and the like refer to cultures that have allowed scientists to view the intestinal epithelium outside of a living organism, while the cells grow and divide. Residing in a Laminin-based matrix known as matrigel (See, e.g., Hughes et al., 2010), epithelial cells can be cultured and grown in structures called Intestinal Organoids, or Enteroids. These budded structures retain the basic crypt-villus morphology of the intestinal epithelium and can be passaged multiple times and exposed to reagents for experimental purposes.

“Dysbiosis” refers to a state of the microbiota of the gut or other body area in a subject, including mucosal or skin surfaces in which the normal diversity and/or function of the ecological network is disrupted. This unhealthy state can be due to a decrease in diversity, the overgrowth of one or more pathogens or pathobionts, symbiotic organisms able to cause disease only when certain genetic and/or environmental conditions are present in a subject, or the shift to an ecological microbial network that no longer provides an essential function to the host subject, and therefore no longer promotes health.

“Pathobionts” refers to any potentially pathological (disease-causing) organism which, under normal circumstances, lives as a symbiont. Pathobionts are also known as opportunistic pathogens.

The terms “treating,” “treatment” or “intervention” refer to the administration or delivery of one or more therapeutic agents, compositions or procedures to a subject who has a disease, condition or disorder or a predisposition toward a disease, condition or disorder, with the purpose to prevent, alleviate, relieve, retard, alter, reverse, remedy, ameliorate, improve, affect, slow or stop the progression, slow or stop the worsening of the disease, condition or disorder, at least one symptom of the disease, condition or disorder, or the predisposition toward the disease, condition or disorder.

As used herein the terms “disease” and “pathologic condition” are used interchangeably, unless indicated otherwise herein, to describe a deviation from the condition regarded as normal or average for members of a species or group (e.g., humans), and which is detrimental to an affected individual under conditions that are not inimical to the majority of individuals of that species or group. Such a deviation can manifest as a state, signs, and/or symptoms (e.g., diarrhea, nausea, fever, pain, blisters, boils, rash, immune suppression, inflammation, etc.) that are associated with any impairment of the normal state of a subject or of any of its organs or tissues that interrupts or modifies the performance of normal functions. A disease or pathological condition may be caused by or result from contact with a microorganism (e.g., a pathogen or other infective agent (e.g., a virus or bacteria)), may be responsive to environmental factors (e.g., malnutrition, industrial hazards, and/or climate), may be responsive to an inherent defect of the organism (e.g., genetic anomalies) or to combinations of these and other factors.

The terms “host,” “subject,” or “patient” as used herein refer to any living organism, including, but not limited to, humans, nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like. The term does not denote a particular age or sex. In a specific embodiment, the subject is human. The subject may be suffering from a dysbiosis, including, but not limited to, gastrointestinal diseases, disorders, and/or conditions (e.g., irritable bowel syndrome, inflammatory bowel disease, obesity, fatty liver disease or other disease, disorder or condition described herein). In the context of the invention, the term “subject” generally refers to an individual who will be administered or who has been administered one or more compositions of the present invention.

The term “metabolic disorder” as used herein, refers to disorders, diseases, and conditions that are caused or characterized by abnormal weight gain, energy use or consumption, altered responses to ingested or endogenous nutrients, energy sources, hormones or other signaling molecules within the body or altered metabolism of carbohydrates, lipids, proteins, nucleic acids or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 or other neurotransmitters or regulatory proteins in the brain) or the like. Some non-limiting examples can be obesity, diabetes, including type II diabetes, insulin-deficiency, insulin-resistance, insulin-resistance related disorders, glucose intolerance, syndrome X, inflammatory and immune disorders, osteoarthritis, dyslipidemia, metabolic syndrome, non-alcoholic fatty liver, abnormal lipid metabolism, cancer, neurodegenerative disorders, sleep apnea, hypertension, high cholesterol, atherogenic dyslipidemia, hyperlipidemic conditions such as atherosclerosis, hypercholesterolemia, and other coronary artery diseases in mammals, and other disorders of metabolism. Disorders also included are conditions that occur or cluster together, and increase the risk for heart disease, stroke, diabetes, and obesity. Having just one of these conditions such as increased blood pressure, elevated insulin levels, excess body fat around the waist or abnormal cholesterol levels can increase the risk of the above mentioned diseases. In combination, the risk for coronary heart disease, stroke, insulin-resistance syndrome, and diabetes is even greater.

As used herein, the term “phenotype” refers to a set of observable characteristics of an individual entity. As example an individual subject may have a phenotype of “health” or “disease”. Phenotypes describe the state of an entity and all entities within a phenotype share the same set of characteristics that describe the phenotype. The phenotype of an individual results in part, or in whole, from the interaction of the entities genome and/or microbiome with the environment.

As used herein, the term “colonization” (e.g., of a host organism) refers to the non-transitory residence of a bacterium or other microscopic organism. As used herein, “reducing colonization” of a host subject's gastrointestinal tract (or any other microbiotal niche) by a pathogenic bacterium includes a reduction in the residence time of the pathogen in the gastrointestinal tract as well as a reduction in the number (or concentration) of the pathogen in the gastrointestinal tract or adhered to the luminal surface of the gastrointestinal tract. Measuring reductions of adherent pathogens may be demonstrated, e.g., by a biopsy sample, or reductions may be measured indirectly, e.g., by measuring the pathogenic burden in the stool of a mammalian host.

As used herein the term “vitamin” refers to any of various fat-soluble or water-soluble organic substances (e.g., vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) essential in minute amounts for normal growth and activity of the body and obtained naturally from plant and animal foods or synthetically made, pro-vitamins, derivatives, and/or analogs.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, the term “antioxidant” is understood to include any one or more of various substances such as beta-carotene (a vitamin A precursor), vitamin C, vitamin E, and selenium) that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

The terms “buffer” or “buffering agents” refer to materials, that when added to a solution, cause the solution to resist changes in pH.

The terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+ charge in solution (i.e., one more proton than electron).

The term “divalent salt” refers to any salt in which a metal (e.g., Mg, Ca, or Sr) has a net 2+ charge in solution.

The terms “chelator” or “chelating agent” refer to any materials having more than one atom with a lone pair of electrons that are available to bond to a metal ion.

The term “solution” refers to an aqueous or non-aqueous mixture.

As used herein, the terms “administration” and “administering” refer to the act of giving a composition of the present invention (e.g., isolated, purified, and/or recombinant p40, or, bacterial cells genetically modified to overexpress p40) to a subject. Exemplary routes of administration to the human body include, but are not limited to, enteral, parenteral, through the eyes (ophthalmic), mouth (oral), skin (transdermal), nose (nasal), lungs (inhalant), oral mucosa (buccal), ear, rectal, by injection (e.g., intravenously, subcutaneously, intraperitoneally, etc.), topically, and the like.

As used herein, the terms “co-administration” and “co-administering” refer to the administration of at least two agent(s) (e.g., a bacterial strain genetically modified to overexpress p40 and one or more bacterial strains not genetically modified, or, a combination of two or more bacterial species, or, a bacterial species and recombinant p40) or therapies to a subject. In some embodiments, the co-administration of two or more agents or therapies is concurrent. In other embodiments, a first agent/therapy is administered prior to a second agent/therapy. In some embodiments, co-administration can be via the same or different route of administration. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone.

A “combination” of two or more bacterial species includes the physical co-existence of the two bacteria, either in the same material or product or in physically connected products, as well as the temporal co-administration or co-localization of the two bacteria.

The terms “pharmaceutically acceptable” or “pharmacologically acceptable,” as used herein, refer to compositions that do not substantially produce adverse reactions (e.g., toxic, allergic or immunological reactions) when administered to a subject.

As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethylene glycol, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a composition of the present invention that is physiologically tolerated in the target subject. “Salts” of the compositions of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compositions of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW4+, wherein W is C1-4 alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na+, NH4+, and NW4+(wherein W is a C1-4 alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compositions of the present invention are contemplated as being pharmaceutically acceptable. However, salts of acids and bases that are non-pharmaceutically acceptable may also find use, for example, in the preparation or purification of a pharmaceutically acceptable composition.

As used herein, the term “at risk for disease” refers to a subject that is predisposed to experiencing a particular disease. This predisposition may be genetic (e.g., a particular genetic tendency to experience the disease, such as heritable disorders), or due to other factors (e.g., environmental conditions, exposures to detrimental compounds present in the environment, etc.). Thus, it is not intended that the present invention be limited to any particular risk (e.g., a subject may be “at risk for disease” simply by being exposed to and interacting with other people), nor is it intended that the present invention be limited to any particular disease (e.g., IBD).

As used herein, the term “kit” refers to any delivery system for delivering materials. In the context of compositions of the invention, such delivery systems include systems that allow for the storage, transport, or delivery of bacteria and/or bacterial products (e.g., p40) and/or supporting materials (e.g., written instructions for using the materials, etc.) from one location to another. For example, kits include one or more enclosures (e.g., boxes) containing the relevant compositions and/or supporting materials. As used herein, the term “fragmented kit” refers to delivery systems comprising two or more separate containers that each contain a sub-portion of the total kit components. The containers may be delivered to the intended recipient together or separately. For example, a first container may contain a composition comprising isolated, purified and/or recombinant p40 of the invention for a particular use, while a second container contains a second agent (e.g., a population of bacteria). Indeed, any delivery system comprising two or more separate containers that each contains a sub-portion of the total kit components are included in the term “fragmented kit.” In contrast, a “combined kit” refers to a delivery system containing all of the components needed for a particular use in a single container (e.g., in a single box housing each of the desired components). The term “kit” includes both fragmented and combined kits.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)2 fragments, and includes immunoglobulins of the following classes: IgG, IgA, IgM, IgD, IgE, and secreted immunoglobulins (slg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)2 fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (e.g., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the terms “epithelial tissue” or “epithelium” refer to the cellular covering of internal and external surfaces of the body, including the lining of vessels and other small cavities. Epithelium is classified into types on the basis of the number of layers deep and the shape of the superficial cells.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5 (carboxyhydroxyl¬methyl) uracil, 5-fluorouracil, 5 bromouracil, 5-carboxymethylaminomethyl 2 thiouracil, 5 carboxymethyl¬aminomethyluracil, dihydrouracil, inosine, N6 isopentenyladenine, 1 methyladenine, 1-methylpseudo¬uracil, 1 methylguanine, 1 methylinosine, 2,2-dimethyl¬guanine, 2 methyladenine, 2 methylguanine, 3-methyl¬cytosine, 5 methylcytosine, N6 methyladenine, 7 methylguanine, 5 methylaminomethyluracil, 5-methoxy-amino¬methyl 2 thiouracil, beta D mannosylqueosine, 5′ methoxycarbonylmethyluracil, 5 methoxyuracil, 2 methylthio N6 isopentenyladenine, uracil 5 oxyacetic acid methylester, uracil 5 oxyacetic acid, oxybutoxosine, pseudouracil, queosine, 2 thiocytosine, 5-methyl-2 thiouracil, 2-thiouracil, 4 thiouracil, 5-methyluracil, N-uracil 5 oxyacetic acid methylester, uracil 5 oxyacetic acid, pseudouracil, queosine, 2-thiocytosine, and 2,6 diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample. As used herein, the term “substantially purified” refers to the removal of from about 70 to 90%, up to 100%, of the contaminants or undesired compounds from a sample or composition.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

“Sequence identity,” “% sequence identity” and the like with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell (e.g., for several days). During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient; in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk-cell lines, the CAD gene that is used in conjunction with CAD-deficient cells and the mammalian hypoxanthine-guanine phosphoribosyl transferase (hprt) gene that is used in conjunction with hprt-cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

Studies have shown that the relationship between gut microbiota and humans is not merely commensal (a non-harmful coexistence), but rather often is a mutualistic, symbiotic relationship. Although animals can survive with no gut microbiota, the microorganisms perform a host of useful functions, such as stimulating immune development, preventing invasion by pathogenic bacteria, regulating the development of the gut, fermenting unused dietary substrates, metabolism of glycans and amino acids, synthesis of vitamins (such as biotin and vitamin K) and isoprenoids, biotransformation of xenobiotics, and directing the host to store fats. Furthermore, it is appreciated that changes in the composition of the gut microbiota have important health effects.

The intestinal epithelial monolayer constitutes a physical and functional barrier between an organism (e.g., a human subject) and the external environment. It regulates nutrient absorption, water and ion fluxes, and represents the first defensive barrier against toxins and enteric pathogens. Epithelial cells are linked together at the apical junctional complex by tight junctions that reduce the extracellular space and the passage of charge entities while forming a physical barrier to lipophilic molecules. Cultured intestinal epithelial cells have been extensively used to study intestinal absorption of newly synthesized drugs and the regulation of tight junctions structure and function. In vitro mild irritants, proinflammatory cytokines, toxins and pathogens, and adverse environmental conditions open tight junctions and increase paracellular permeability, an effect often accompanied by immune activation of the enterocytes. Conversely, inhibition of proinflammatory cytokines, exposure to growth factors and probiotics, among others, exert a protective effect. Impaired barrier function results from activation of signalling pathways that lead to alteration of junctional proteins expression and/or distribution. In vivo, intestinal barrier dysfunction is associated with various intestinal and non-intestinal disorders including inflammatory bowel disease, celiac disease, metabolic syndromes, fatty liver disease, and diarrhoeal infection.

Thus, the intestinal epithelium forms the protective barrier and host defense against the harmful luminal microenvironment with selective permeability and absorption of nutrients. The epithelium is covered by a single-cell layer composed of different subtypes of specialized intestinal epithelial cells (IECs) including absorptive cells, goblet cells, enteroendocrine cells, Paneth cells, M cells, cup cells, and Tuft cells. These subsets of IECs are functionally different and essential to maintain intestinal homeostasis by separating the intestinal lumen from the underlying lamina propria and by controlling the crosstalk between microbiota and subjacent immune cells. Thus, a dysregulation of the differentiation system for correct IEC formation has a crucial role in the pathogenesis of inflammatory bowel disease (IBD).

The epithelial monolayer is the main component of the epithelial barrier and its ability to act as a protective physical barrier is mediated by the formation of a web of tight junctions (TJs) that regulate the paracellular permeability and barrier integrity, production of mucus layer covering the luminal surface of the epithelium, and recognition of pathogens and production of antimicrobial peptides (AMPs) to ensure effective immunity. TJs seal the paracellular space between epithelial cells and separate the cell membrane into apical and basolateral domains, thus forming a physical barrier against foreign antigens. Altered expression and structural changes of the intestinal TJ proteins are closely associated with the development of IBD. Moreover, several pro-inflammatory cytokines, such as tumor necrosis factor (TNF)-alpha and interferon-γ, have been shown to increase TJ permeability and to induce apoptosis of IECs leading to the loss of epithelial barrier function and induction of epithelial damage and ulcers that are present in mucosal inflammation. As mentioned above, abnormal intestinal permeability has been linked with various intestinal and non-intestinal disorders including inflammatory bowel disease (IBD), metabolic syndromes, fatty liver disease, celiac disease, and diarrhoeal infection. Clinically, probiotics have been shown to improve intestinal barrier function in experimental animals and reduce symptoms of IBS patients.

Using an enteroid model system, experiments conducted during development of embodiments of the invention identified that the supernatant of Lactobacillus rhamnosus GG (LGG) prevented IFN-γ-induced epithelial barrier damage (See, e.g., Examples 1 and 2). The beneficial effects occurred independently of immune modulating effects of the probiotic (See, e.g., Example 4). Additional experiments were carried out in an effort to identify and characterize what constituent(s) of the supernatant was/were responsible for the prevention of IFN-γ-induced epithelial barrier damage. In particular, experiments were carried out during development of embodiments of the invention that identified and characterized a protein secreted by LGG, p40, one of the major bands observed in SDS-PAGE analyses of concentrated LGG supernatant (See, e.g., Examples 3-8).

A strategy to synthesize and characterize p40 was generated. As described in the Examples, the coding sequence for p40 was PCR amplified from LGG genomic DNA and subcloned into the PET28b+ expression vector. The protein was produced using the E. coli strain BL21 (DE3)/pLysS and purified by nickel nitrilotrialetic acid agarose and fast protein liquid chromatography. Using a reductionist approach, human colonoid, a 3D structure grown from human colonic biopsies, was used. Fecal supernatant (FSN) from IBS-D patients was used to induce epithelial barrier damage resulting in a 40% and 50% reduction in gene expression of occludin and ZO-1. (p<0.05) (See, e.g., Example 7). These changes were accompanied by a 28% increase in DNA methyltransferase (DNMT1) protein expression and a 40% decrease in acetyl-histone H3 (ep300) protein expression. Pretreatment with p40 prevented the changes in DNMT1 and ep300 and normalized the protein expression of Z01 and occludin (See, e.g., Examples 7 and 8). Thus, although an understanding of a mechanism is not needed to practice the present invention, and while the invention is not limited to any particular mechanism of action, in some embodiments, administration of p40 to a subject acts via epigenetic regulatory pathways to increase the synthesis of junction proteins (e.g., Z01 and occludin).

In separate studies, an in-outside human colonoid structures was used to evaluate the permeability of the epithelium. Human colonoids were injected with the fluorescence dye FD4 and images were obtained at different time points (See, e.g., Example 5). Under control conditions, the human colonoids retained 70% of FD4 over 10 h. Treatment of the colonoids with IBS-FSN impaired permeability resulting in 10% retention of the dye at 10 h. These changes were prevented by p40. Silencing DNMT1 gene in human colonoids with specific siRNA also prevented the leakage of dye evoked by IBS-FSN. Thus, as documented in the Examples, p40 stimulated the production of tight junction proteins Z01 and occludin, and it also improved mucosa permeability. Accordingly, the invention provides compositions comprising p40 (e.g., those described herein) that find use in enhancing junction protein synthesis (e.g., the synthesis of junction proteins Z01 and occludin) and to enhance mucosa protection (e.g., in a subject that would benefit from such administration (e.g., a subject with inflammatory bowel disease (IBD), irritable bowel syndrome (IBS), or other gastrointestinal disorder, disease, or condition)).

Additional, in vivo experiments were conducted during development of embodiments of the invention in order to confirm the in vitro organoid discoveries described in Examples 1-8. Specifically, a rat model was generated and utilized in which intracolonic infusion of fecal supernatant from symptomatic Irritable Bowel Syndrome (IBS) patients was administered to naïve rats and shown to induce submucosal inflammation, intestinal barrier dysfunction and development of visceral hypersensitivity (see Zhou et al., J Clin Invest 2018: 128(1) 267-280). This was mediated by elevated fecal levels of lipopolysaccharide (LPS) due to gut dysbiosis. This rodent model of gut inflammation was used to examine the ability of P40 secreted by Lactobacillus rhamnosus GG (LGG) to ameliorate colonic inflammation and visceral hypersensitivity. As described in Example 9, experiments conducted during development of embodiments of the invention discovered and characterized for the first time that p40 promoted in vivo junction protein expression, maintained mucosal epithelial barrier function and prevented mucosal inflammation evoked by fecal supernatants from symptomatic IBS patients. P40 also prevented the development of visceral hypersensitivity. While it is not necessary to understand the mechanism of action in order to practice the present invention, and while the present invention is not limited to any particular mechanism, in one embodiment, topical application of P40 acts directly on epithelial cells to prevent barrier dysfunction and mucosa inflammation (e.g., induced by fecal supernatant from IBS patients).

Various studies have been carried out, with mixed results, using probiotics in an attempt to improve IBS symptoms. Probiotics have also been suggested to modulate the immune system. Although an understanding of a mechanism is not needed to practice the present invention, and while the invention is not limited to any particular mechanism of action, in some embodiments, administration of a composition of the invention (e.g., one or more species of Lactobacilli genetically modified to overexpress p40 used independently and/or combined with other microbiota (e.g., that are not genetically modified), p40 isolated and/or purified from Lactobacilli genetically modified to overexpress p40, recombinant p40, and/or cell lysate(s) from one or more species of Lactobacilli genetically modified to overexpress p40) provides a therapeutically effective amount (e.g., to prevent epithelial barrier damage, to enhance junction protein synthesis, and/or to enhance mucosa protection) of p40 to a subject (e.g., an amount that is not obtained by a subject without administration of the composition of the invention (e.g., an amount obtained by a conventional diet and/or conventional probiotic treatment)). Thus, in contrast to conventional probiotic use where there is insufficient probiotic concentrations ingested by subjects to produce significantly beneficial effect, compositions of the invention can be produced in large quantities (e.g. via genetically engineering (e.g., engineering bacteria and using the engineered bacteria and/or subcomponents thereof (e.g., p40)) and are useful in methods described herein to produce significantly beneficial effects in a subject (e.g., to prevent epithelial barrier damage, to enhance junction protein synthesis, and/or to enhance mucosa protection).

Accordingly, the invention provides methods and compositions for treating (e.g., therapeutically or prophylactically) gastrointestinal diseases, disorders, and conditions. In particular, in one aspect the invention provides compositions comprising Lactobacillus (e.g., Lactobacillus rhamnosus or other Lactobacillus species) p40 protein (e.g., isolated, recombinant, or overexpressed p40 protein) and methods of using same for treating a gastrointestinal disease, disorder, or condition. Another aspect provides methods and compositions for treating a subject with a gastrointestinal disease, disorder, or condition by administering to the subject a composition that includes isolated, recombinant, or overexpressed Lactobacillus p40 protein. Yet another aspect of the invention includes pharmaceutical compositions (e.g., for altering microbiota in a subject or for treating a gastrointestinal disease, disorder, or condition in a subject) comprising a therapeutically effective amount of isolated, recombinant, or overexpressed Lactobacillus p40 protein.

In one aspect, the invention provides compositions comprising Lactobacillus (e.g., Lactobacillus rhamnosus (e.g., Lactobacillus rhamnosus GG) or other Lactobacillus species) p40 protein (e.g., for treating a gastrointestinal disease, disorder, or condition in a subject). The composition can be a cell based composition comprising bacterial cells modified (e.g., genetically engineered) to express (e.g., overexpress) or to harbor Lactobacillus p40. The composition may also be a cell based composition comprising bacterial cells administered with isolated (e.g., recombinant) Lactobacillus p40. The composition can also be a cell based composition comprising bacterial cells and further including one or more bacterial strains modified (e.g., genetically) to express (e.g., overexpress) Lactobacillus p40. For example, in one embodiment, a cell based composition comprises purified microbiota from phyla such as Bacteroidetes, Proteobacteria, Firmicutes and Verrucomicrobia or orders such as Bacteroidales, Enterobacteriales, Clostridiales, and Verrucomicrobiales, or genera such as Alistipes, Escherichia, Clostridium, Lactobacillus or Akkermansia that have been modified (e.g., genetically engineered) to overexpress Lactobacillus p40. In another embodiment, the microbiota are combined with isolated, purified, and/or recombinant p40. In still another embodiment, the microbiota are combined with one or more strains of Lactobacillus that have been modified (e.g., genetically engineered) to overexpress p40. The invention is not limited by the specific species of Lactobacillus p40 used. Indeed, any Lactobacilli p40 may be used including, but not limited to, L. acidophilus, L. brevis, L. bulgaricus, L. casei, L. fermentum, L. gasseri, L. paracasei, L. plantarum, L. reuteri, L. rhamnosus, L. salivarius, and/or L. sporogenes. In a preferred embodiment, L. rhamnosus (e.g., LGG) p40 is used. In one embodiment, p40 from two or more different species of Lactobacilli are used. P40 may be isolated and/or purified p40. In another embodiment, p40 is recombinant p40.

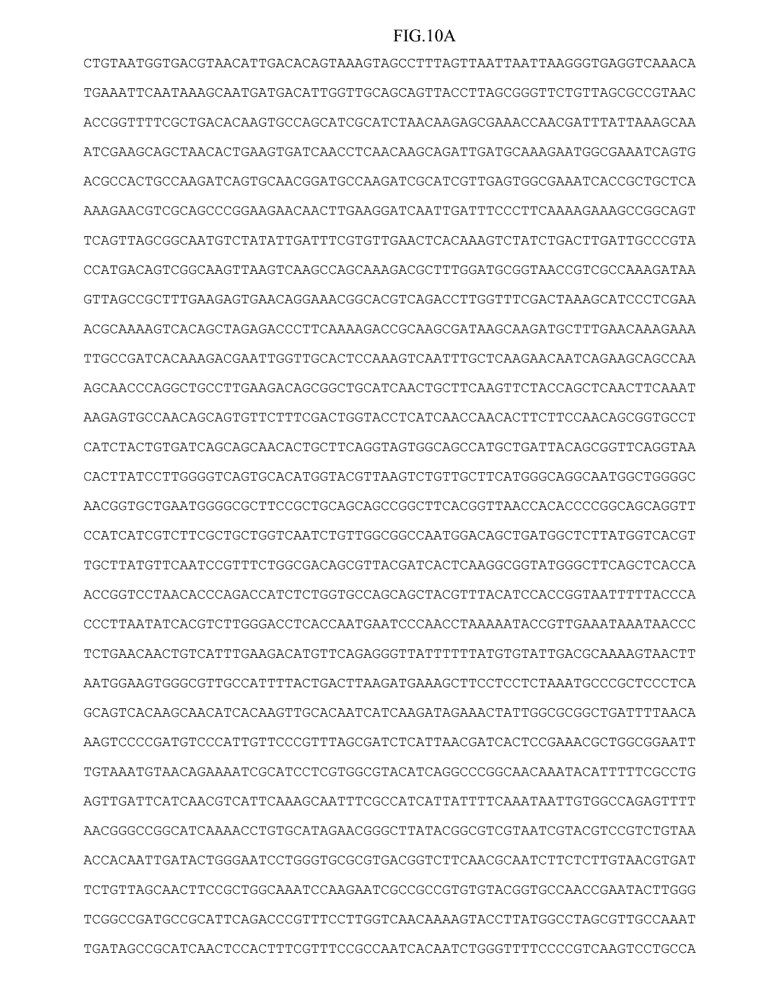

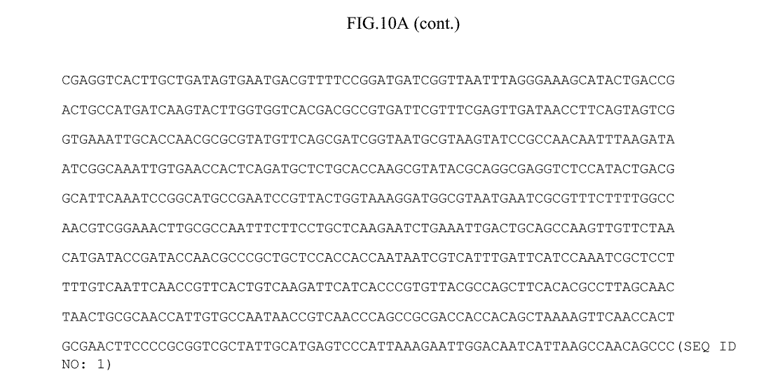

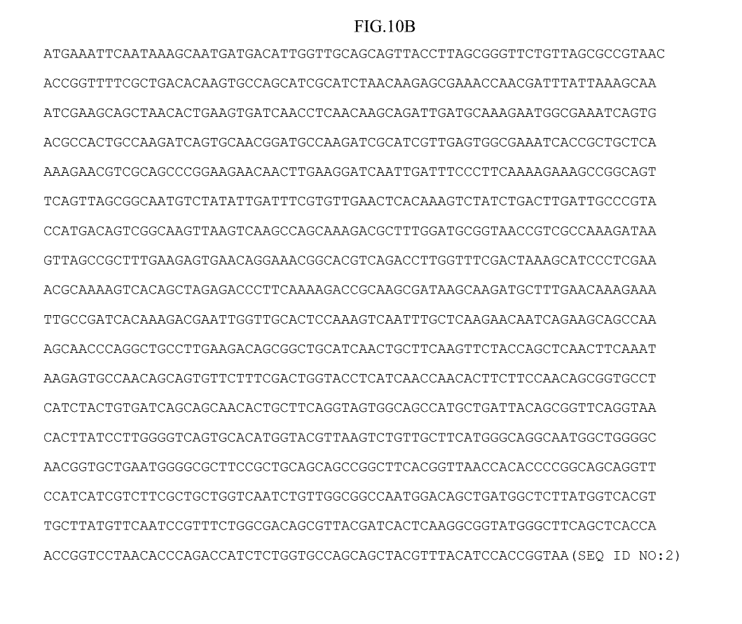

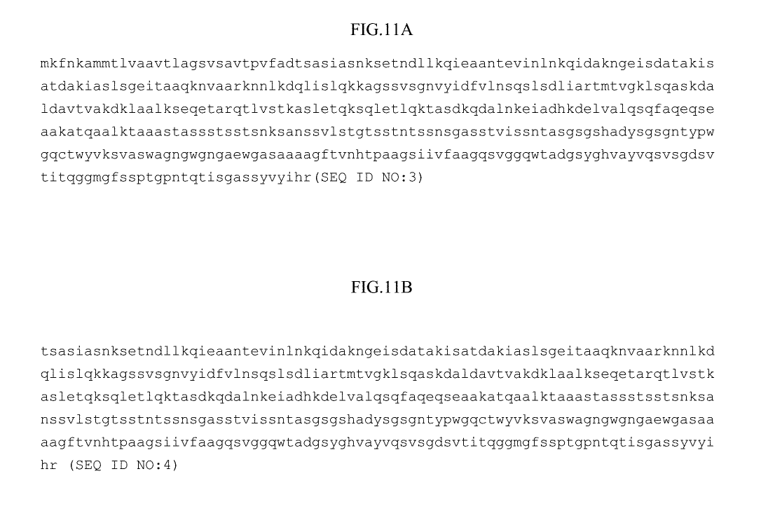

The invention is not limited by the type or form of p40 used. For example, in one embodiment, p40 expressed or harbored in a cell is free p40 (e.g., p40 polypeptide or oligomers of p40). Full length p40, or a biologically active fragment thereof (e.g., that prevents epithelial barrier damage, enhances junction protein synthesis, and/or enhances mucosa protection) may be used. For example, in one embodiment, all or a portion of the nucleic acid sequence of SEQ ID NO. 1 (See FIG. 10A) is used (e.g., to express (e.g., using an expression vector to express in vivo or in vitro) p40). In another embodiment, all or a portion of the nucleic acid sequence of SEQ ID NO. 2 (See FIG. 10B) is used (e.g., to express (e.g., using an expression vector to express in vivo or in vitro) p40). Any p40 peptide, or fragment thereof, that is biologically active (e.g., that prevents epithelial barrier damage, enhances junction protein synthesis, and/or enhances mucosa protection) finds use in the invention. For example, in some embodiments, p40 used in the compositions and methods of the invention comprises an amino acid sequence having at least 75%, 80%, 85%, 90%, 95%, 97%, 98%, 99% or more sequence identity to the amino acid sequence of SEQ ID NO: 3 (See FIG. 11A). In another embodiment, p40 used in the compositions and methods of the invention comprises an amino acid sequence having at least 75%, 80%, 85%, 90%, 95%, 97%, 98%, 99% or more sequence identity to the amino acid sequence of SEQ ID NO: 4 (See FIG. 11B).

The invention is not limited to any particular fragment of p40. Indeed, any fragment of p40 that is known or shown to be biologically active (e.g., that prevents epithelial barrier damage, enhances junction protein synthesis, and/or enhances mucosa protection) can be used. Furthermore, fragments of p40 can be assessed and identified as biologically active using methods described herein and/or methods known in the art. In some embodiments, full length p40, or a biologically active fragment thereof, is used (e.g., is co-administered) with a one or more other biologically active substances (e.g., to prevent epithelial barrier damage, to enhance junction protein synthesis, and/or to enhance mucosa protection) in a composition and/or method of the invention. Any Lactobacilli p40 may be used including, but not limited to, L. acidophilus, L. brevis, L. bulgaricus, L. casei, L. fermentum, L. gasseri, L. paracasei, L. plantarum, L. reuteri, L. rhamnosus, L. salivarius, and/or L. sporogenes. In a preferred embodiment, L. rhamnosus (e.g., LGG) p40 is used.

A composition of the invention may include Lactobacilli genetically modified to overexpress p40 (e.g., used independently and/or combined with other microbiota (e.g., that are not genetically modified)). A composition of the invention may include p40 isolated and or purified from Lactobacilli (e.g., one or more species of Lactobacilli genetically modified to overexpress p40). A composition of the invention may comprise a cell lysate (e.g., prepared from one or more species of Lactobacilli genetically modified to overexpress p40). In a further embodiment, the invention provides a composition (e.g., for use in a therapeutic method described herein) comprising a cell lysate harvested from bacterial cells (e.g., one or more strains of Lactobacilli genetically modified to overexpress p40) and also includes one or more bacterial cell species (e.g., one or more microbiota from phyla such as Bacteroidetes, Proteobacteria, Firmicutes and Verrucomicrobia or orders such as Bacteroidales, Enterobacteriales, Clostridiales, and Verrucomicrobiales, or genera such as Alistipes, Bacteroides, Escherichia, Clostridium, Lactobacillus or Prevotella, Parabacteroides and/or Ruminococcus). In another embodiment, the invention provides a composition (e.g., for use in a therapeutic method described herein) comprising a cell lysate (e.g., harvested from one or more species of Lactobacilli genetically modified to overexpress p40), one or more microbiota from phyla such as Bacteroidetes, Proteobacteria, Firmicutes and Verrucomicrobia or orders such as Bacteroidales, Enterobacteriales, Clostridiales, and Verrucomicrobiales, or genera such as Alistipes, Bacteroides, Escherichia, Clostridium, Lactobacillus or Prevotella, Parabacteroides and/or Ruminococcus, and includes isolated and/or recombinant p40.

Any expression vector system known in the art may be utilized for expression of a p40 nucleic acid sequence (e.g., encoding full length p40, a fragment thereof, and/or dimer, trimer, tetramer, or higher order oligomer complexes thereof) in a bacterial cell. In one embodiment, a vector comprising a nucleic acid sequence encoding p40, operably linked to a promoter and expression/control sequences necessary for expression thereof is used. p40 nucleic acid sequence may comprise the entire coding sequence or any portion thereof that encodes a biologically active portion thereof p40 expressed or harbored in a cell may comprise the entire p40 polypeptide or any portion thereof capable of preventing epithelial barrier damage, enhancing junction protein synthesis, and/or enhancing mucosa protection that would otherwise not occur in the absence of the p40 polypeptide (e.g., identified and/or determined using compositions and methods disclosed herein (e.g., in the Examples)). p40 may be expressed or present as a monomer, dimer, trimer, tetramer, or higher order oligomer complexes (e.g., 5, 6, 7, 8, 9, 10, 12, 15, 20, 25, 30 or more). For example, in one embodiment, all or a portion of the nucleic acid sequence of SEQ ID NO: 1 or SEQ ID NO: 2 is used to introduce p40 into cells (e.g., for expression of a p40 monomer, dimer and/or trimer therein). Any expression construct available in the art may be used to express p40 in cells including those described herein.

The invention also provides methods for treating (e.g., therapeutically and/or prophylactically) a gastrointestinal disease, disorder, or condition (e.g., via administration of a composition comprising Lactobacillus (e.g., Lactobacillus rhamnosus or other Lactobacillus species) p40 protein (e.g., isolated, recombinant, or overexpressed p40 protein)). For example, in one embodiment, the invention provides a method of treating a subject with a gastrointestinal disease, disorder, or condition by administering to the subject a therapeutically effective amount of a composition comprising isolated, recombinant, and/or overexpressed Lactobacillus p40 protein (e.g., one or more species of Lactobacilli genetically modified to overexpress p40 (e.g., used independently and/or combined with other microbiota (e.g., that are not genetically modified))).

The invention is not limited by the type of gastrointestinal disease, disorder, or condition that can be treated (e.g., therapeutically and/or prophylactically) using compositions and methods of the invention. In one embodiment, the disorder is irritable bowel syndrome. In another embodiment, the disease is inflammatory bowel disease In still another embodiment, the condition is obesity. In another embodiment, the condition is fatty liver disease. In one embodiment, the subject is one with a disease, disorder, or condition that would benefit from maintaining and/or improving mucosal epithelial barrier function (e.g., via administration of a composition of the invention (e.g., a composition comprising isolated, recombinant, and/or overexpressed Lactobacillus p40 protein (e.g., one or more species of Lactobacilli genetically modified to overexpress p40 (e.g., used independently and/or combined with other microbiota (e.g., that are not genetically modified))))). In another embodiment, the subject is one with a disease, disorder, or condition that would benefit from the prevention of or amelioration of intestinal mucosal tight junction damage (e.g., via administration of a composition of the invention (e.g., a composition comprising isolated, recombinant, and/or overexpressed Lactobacillus p40 protein (e.g., one or more species of Lactobacilli genetically modified to overexpress p40 (e.g., used independently and/or combined with other microbiota (e.g., that are not genetically modified))))).

The invention also provides methods of manufacturing any one of the compositions comprising p40 of the invention, or a combination thereof, described herein.

The invention further provides methods of using one or more of the compositions of the invention for treating (e.g., therapeutically and/or prophylactically) a gastrointestinal disease, disorder, or condition in a subject (e.g., an IBD patient, and IBS patient, or other subject described herein). In a further embodiment, following administration of a composition of the invention to a subject/patient, one or more physiologic responses in the subject is detected (e.g., wherein the one or more physiologic responses are not detected the patient prior to administering the composition). In one embodiment, because the amount of p40 administered to a patient can be controlled and/or monitored (e.g., due to the exact amount of recombinant p40 present in a composition of the invention being known and/or knowable), the invention also provides methods of tailoring the amount of p40 administered to a subject (e.g., so as to provide customized care based upon a subject's disease, disorder, and/or condition).

Compositions of the invention may include a “therapeutically effective amount,” or an “effective amount.” A “therapeutically effective amount” refers to an amount effective, at dosages and for periods of time necessary, to achieve the desired therapeutic result. A therapeutically effective amount of a composition may vary according to factors such as the disease state, age, sex, and weight of the individual, and the ability of the composition to elicit a desired response in the individual. A therapeutically effective amount is also one in which any toxic or detrimental effects of the composition are outweighed by the therapeutically beneficial effects. In an exemplary embodiment, a therapeutically effective amount of a composition of the invention is one in which the amount increases a relative abundance of p40 in a subject.

The dosage of the compositions can be dependent on the types or source of p40 (e.g., one or more species of Lactobacilli genetically modified to overexpress p40 used independently and/or combined with other microbiota (e.g., that are not genetically modified), p40 isolated and/or purified from Lactobacilli genetically modified to overexpress p40, recombinant p40, and/or cell lysate(s) from one or more species of Lactobacilli genetically modified to overexpress p40) used as and/or in the composition. Alternatively, a dosage can also be determined based on a relative abundance of one or more microbiota present in the subject. The dosage can also be determined by additional treatments or therapeutic interventions, such as procedures like administration of a composition or agent like a supplement, pharmaceutical therapy, pharmaceutical administration, electrical stimulation of nerves that innervate at least a portion of the gastrointestinal tract, therapies impacting circadian rhythms, bile acid modulation, intestinal mucus production and metabolism, gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, duodenal endoluminal barrier, or similar manipulations of the gastrointestinal tract.

In one embodiment, the composition is effective to alter the relative abundance of p40 (e.g., in the gastrointestinal tract of a subject). In another embodiment, the composition is effective to increase a relative abundance of one or more strains of Lactobacilli (e.g., wild type and/or genetically modified to overexpress p40) in a subject. In one embodiment, the composition can increase or decrease a relative abundance of a specific strain of microbiota from phyla such as Bacteroidetes, Proteobacteria, Firmicutes, Tenericutes, and Verrucomicrobia or orders such as Bacteroidales, Enterobacteriales, Erysipelotrichales, Clostridiales and Verrucomicrobiales or genera such as Alistipes, Escherichia, Clostridium, Allobaculum, and Akkermansia, in a subject. A composition of the invention can be effective to alter microbiota to mimic microbiota from normal, healthy subjects of similar weight, age, gender, race, etc.

Dosage regimens may be adjusted to provide the optimum desired response (e.g., a therapeutic or prophylactic response). For example, a single bolus may be delivered, several divided doses may be delivered over time or the dose may be proportionally reduced or increased as indicated by the exigencies of the therapeutic situation. It is especially advantageous to formulate parenteral compositions in dosage unit form for ease of delivery and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of individuals.

Exemplary, non-limiting dosages of a composition when employed in a method of the invention (e.g., when using genetically modified bacteria) can be in the range from about 0.001 to about 100 mg/kg body weight per day, from about 0.01 to about 50 mg/kg body weight per day, such as from about 0.05 to about 10 mg/kg body weight per day, delivered in one or most doses, such as from 1 to 3 doses. In an exemplary embodiment, the composition includes substantially purified bacteria (e.g., genetically modified bacteria alone or in combination with other, non-genetically modified bacteria) in the range of about 0.01 to about 50 mg/kg body weight per day, delivered in one to three doses. The exact dosage will depend upon the frequency and mode of delivery, the gender, age, weight and general condition of the subject treated, the nature and severity of the condition treated, any concomitant diseases to be treated and other factors evident to those skilled in the art.

Compositions of the invention can be delivered or administered by a variety of methods known in the art. The terms “delivery,” “deliver,” “administration” and “administer” are used interchangeable herein. As will be appreciated by a skilled artisan, the route and/or mode of delivery will vary depending upon the desired results. In one embodiment, a composition is delivered perorally. In another embodiment, a composition is delivered orally. In another embodiment, delivery includes methods and combinations for delivery to the gut.

The invention also provides methods for delivering a composition of the invention (e.g., one or more species of Lactobacilli genetically modified to overexpress p40 used independently and/or combined with other microbiota (e.g., that are not genetically modified), p40 isolated and/or purified from Lactobacilli genetically modified to overexpress p40, and/or cell lysate(s) from one or more species of Lactobacilli genetically modified to overexpress p40) to a subject (e.g., to a target location within the subject). For example, p40 can be recombinantly produced (e.g., in large quantities using genetic engineering techniques (e.g., those described herein and/or known in the art)) and delivered to patients. The invention is not limited by the type or route of administration. In some embodiments, a composition of the invention is delivered to a subject (e.g., an IBS or IBD patient) using timed released capsules. In another embodiment, genetically engineered bacteria (e.g., one or more strains of lactobacilli modified to overexpress p40) are generated and are used in the same way a conventional probiotic is used and/or administered to a subject (e.g., in order to deliver elevated levels of p40 to a subject). In another embodiment, the composition is directly delivered to at least a stomach, a small intestine, and/or a large intestine of the subject. Compositions can also be formulated for oral delivery. Delivery methods can also include administering a composition of the invention to a subject and also performing a surgical procedure selected from gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, duodenal endoluminal barrier, similar manipulations of the gastrointestinal tract, and other gastrointestinal bariatric and metabolic procedures. Delivery methods also include administering an additional agent, such as an antibiotic and/or an osmotic laxative, to the subject before, concurrent with, and/or after administration of the composition.

The compositions can be formulated in a variety of forms. These include, for example, liquid, semi-solid and solid dosage forms, such as liquid solutions (e.g., injectable and infusible solutions), dispersions or suspensions, tablets, pills, powders, liposomes, suppositories, and other formulations. The compositions can also be formulated for high drug concentrations. The compositions can further be sterile and stable under the conditions of manufacture and storage. Sterile injectable solutions can be prepared by incorporating the compositions in a required amount of an appropriate solvent with one or a combination of ingredients enumerated above, as required, followed by filtered sterilization.

Compositions of the invention may be formulated as a pharmaceutical composition that includes a pharmaceutically acceptable carrier and administered alone or co-administered with one or more other pharmaceutical compositions. A therapeutically effective amount of such compositions can be contained in food, drink, dietary supplement, and/or food additive to be administered and/or consumed by a subject.

Exemplary forms of the compositions can depend on the intended mode of delivery and therapeutic application. In one embodiment, the composition is formulated for oral delivery. Some compositions can be in the form of pill-based delivery, such as disclosed in U.S. patent application Ser. No. 12/976,648 entitled “Pill Catcher,” filed Dec. 22, 2010, and delayed release methods. In one embodiment, the pill-based delivery can be part of the system that allows the delivery to occur at a precise location within the gastrointestinal tract. In another embodiment, the compositions can be formulated in a delayed release formulation. In another embodiment, the composition can be encapsulated in a coating that does not begin to degrade until it exits the stomach of a patient. In another embodiment, the composition can be prepared with a carrier that protects the composition against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art. See, e.g., Sustained and Controlled Release Drug Delivery Systems, J. R. Robinson, ed., Marcel Dekker, Inc., New York, 1978. “Sustained release” refers to release of a composition or an active compound thereof over a prolonged period of time relative to that achieved by delivery of a conventional formulation of the composition.

A composition of the invention may also be formulated and delivered in an activatable form, such as formulating a composition of the invention in a dormant or inactive state, such as, a lyophilized state. In a combination composition, a composition of the invention (e.g., Lactobacilli engineered to overexpress p40 individually or in combination with one or more other bacterial strains) may be in a dormant or inactive state or the compounds or agents that foster microbiota can be inactive. In an exemplary embodiment, the composition is formulated to include at least one of a dormant or inactive microbiota and inactive compounds or agents that foster microbiota.

The disclosed compositions and combination compositions can also be formulated as a food, drink, dietary supplement, and/or additive. Such compositions are those that are suitable for human and/or animal consumption. A skilled artisan will be readily aware of specific formulations which can be used in oral or ingestible formulations and are considered suitable for human and/or animal administration.

Consumable compositions can be formulated to include a sweetener(s), a stabilizer(s) or binder(s), a humectant(s), and/or natural and/or artificial flavors. The compositions may also include natural and/or artificial colors and preservatives. In one implementation, the compositions may include mono-saccharides, di-saccharides and poly-saccharides such as but not limited to, sucrose (sugar), dextrose, maltose, dextrin, xylose, ribose, glucose, mannose, galactose, sucromalt, fructose (levulose), invert sugar, corn syrups, maltodextrins, fructo oligo saccharide syrups, partially hydrolyzed starch, corn syrup solids, polydextrose, soluble fibers, insoluble fibers, natural cane juice, gelatin, citric acid, lactic acid, natural colors, natural flavors, fractionated coconut oil, carnauba wax, or combinations thereof.

In one embodiment, administration of a composition of the invention and optionally one or more other therapeutic agents results in enhanced therapeutic efficacy and/or potency relative to administration of the composition of the invention or the one or more other therapeutic agents alone. The invention is not limited by the route or frequency of administration of a composition of the invention. Any suitable route of administration can be used to introduce a composition of the invention into a subject including, but not limited to, enteral, parenteral, intravenous, subcutaneous administration, or other route or means described herein and/or known in the art. For example, local or systemic delivery can be accomplished by administration comprising administration of the combination into body cavities, by parenteral introduction, comprising intramuscular, intravenous, intraportal, intrahepatic, peritoneal, subcutaneous, and/or intradermal administration. A composition of the invention may be administered proceeding, following, or in lieu of other treatments and/or therapies for treating and/or preventing IBD, IBS, or other gastrointestinal disease, disorder and/or condition. In one embodiment, following administration, a response (e.g., epithelial barrier leakage and/or permeability) is detected and/or assessed wherein the response is not detected/assessed in a subject prior to administering the composition. In another embodiment, subsequent to detecting one or more responses in a subject, treatment of the patient is modified based on the status of the response(s) detected/assessed in the patient.

A composition of the invention can be delivered to target regions and/or structures within the subject. Regions that can be targeted within the gastrointestinal tract can include, but are not limited to, the stomach, biliopancreatic limb, Roux limb, common limb, ileum, cecum, or colon. Structures can be targeted that constitute differentiated ecological niches with specific pH range, temperature, moisture, and metabolite content.

A region can include but is not limited to a region within the gastrointestinal tract. In an exemplary embodiment, the delivery is targeted to an oral cavity, stomach, biliopancreatic limb, Roux limb, common limb, small intestine, ileum, cecum, large intestine, or colon of a gastrointestinal tract. The delivery can also be targeted to one or more tissues in a subject. The tissues can include any tissue in a gastrointestinal tract, such as a stomach, biliopancreatic limb, Roux limb, common limb, small intestine, ileum, cecum, large intestine, or colon.

A composition of the invention can be delivered before, current with or after a therapeutic treatment, such as procedures like surgery, pharmaceutical therapy and/or administration, electrical stimulation of nerves that innervate at least a portion of the gastrointestinal tract, therapies impacting circadian rhythms, bile acid modulation, intestinal mucus production and metabolism, gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, duodenal endoluminal barrier, or similar manipulations of the gastrointestinal tract. In one embodiment, a composition of the invention is delivered at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 days or more before the treatment. In another embodiment, a composition of the invention is delivered at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 days or more after the treatment. In yet another embodiment, a composition of the invention is delivered concurrently with the therapeutic treatment. In another embodiment, a composition of the invention is delivered for a treatment period that lasts until the desired outcome (e.g., enhancement of junction protein synthesis (e.g., the synthesis of junction proteins Z01 and occludin) and/or enhancement mucosa protection)). Delivery of a composition of the invention can be repeated one or more times. Repeated delivery of a composition of the invention can be one or more times before and/or after a separate treatment (e.g., pharmaceutical treatment). A repeated delivery can be in the same or different manner (e.g., route) to an initial delivery.

A composition of the invention can be administered with other molecules and/or agents including, but not limited to, therapeutic, prophylactic, or diagnostic agents such as small molecules, nucleic acids, proteins, prebiotics like polypeptides, prebiotics including bacterial components such as bacterial cell wall components such as peptidoglycan, bacterial nucleic acids such as DNA and RNA, bacterial membrane components, and bacterial structural components such as proteins, carbohydrates, lipids, lipoproteins, glycolipids and glycoproteins, bacterial metabolites, organic acids, inorganic acids, bases, proteins and peptides, enzymes and co-enzymes, amino acids and nucleic acids, carbohydrates, lipids, glycoproteins, lipoproteins, glycolipids, vitamins, bioactive compounds, metabolites containing an inorganic component, and small molecules such as nitrous molecules or molecules containing a sulphurous acid, resistant starch, potato starch or high amylose starch, modified starches (including carboxylated starches, acetylated, propionated, and butyrated starches), non-digestible oligosaccharides such as fructooligosaccharides, glucooligosaccharides, xylooligosaccharides, galactooligosaccharides, arabinoxylans, arabinogalactans, galactomannans, polydextrose, oligofructose, inulin, derivatives of these, but not excluding other oligosaccharides able to exert prebiotic effects, other soluble fibers, and combinations thereof. In one embodiment, the agent delivered is a small molecule delivered that has low oral bioavailability and acts on a microbial niche of the host's gut. Low oral bioavailability is generally undesirable in drugs, since absorption through the intestine is an objective of most oral therapies.

Certain exemplary embodiments will now be described to provide an overall understanding of the principles of the structure, function, manufacture, and use of the therapeutics and methods disclosed herein. One or more examples of these embodiments are illustrated in the accompanying drawings. Those skilled in the art will understand that the therapeutics and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

All publications, patents and patent applications cited herein are hereby incorporated by reference in their entirety. As used in this specification and the appended claims, the singular forms a,” “an,” and “the” include plural references unless the content clearly dictates otherwise. The terms used in this invention adhere to standard definitions generally accepted by those having ordinary skill in the art.

### EXAMPLES

The following examples serve to illustrate certain preferred embodiments and aspects of the present invention and are not to be construed as limiting the scope thereof.

### Example 1

### Materials & Methods

Subcloning, expression, purification of recombinant Lactobacillus rhamnosus GG (LGG) soluble protein p40 from LGG supernatant. The coding sequence for p40, without the N-terminal signal peptide sequence, was PCR amplified from LGG genomic DNA using pairs of flanking 59- and 39-end oligoprimers, one containing an EcoRI restriction site, and another containing an XhoI restriction site. PCR fragments were cleaved with EcoRI and XhoI restriction endonucleases and subcloned into the pET28b+ expression vector. The resulting recombinant plasmids were propagated in the E. coli strain BL21 (DE3)/pLysS for the expression of p40. E. coli cells having the plasmid were grown at 37° C. in LB-kanamycin(50 mg/ml) broth. Cells were then recovered by centrifugation and broken by sonication in lysis buffer. The protein was purified by nickel nitrilotrialetic acid agarose and fast protein liquid chromatography. Purified p40 was buffer exchanged to 10 mM Tris-HCl with a desalting column and then concentrated. Eluted proteins were separated by SDS-PAGE and stained with Colloidal Blue Staining to show recombinant purified p40 protein.

Generation of P40 specific polyclonal antibody. Polyclonal antibodies against recombinant p40 and LGG supernatant were generated and purified by Pacific Immunology company, and used in Western blot analysis. Western blot analysis was used to show P40 antibody against recombinant p40, p40 in LGG supernatant and after immunodepletion of p40 from LGG supernatant and recombinant p40.

Mice enteroids culture. Mice ileum was excised, opened longitudinally, and washed with cold PBS. The ileum was cut into small pieces and incubated in ice-cold PBS containing 3 mM EDTA for 30 min. After being rinsed once with ice-cold PBS to remove EDTA, the fragments were vigorously shaken in PBS. The supernatant was collected and passed through a 70 μm cell strainer. Crypts were separated from suspended single cells by centrifugation (200 g, 1 min). The pellet was resuspended with DMEM/F12 and mixed with Matrigel for plating. Culture medium composed of AdDMEM/F12 supplemented with 500 ng/ml RSPO1, 100 ng/ml Noggin-conditioned, and 100 ng/ml epidermal growth factor (EGF) was added and changed every 2-4 days.

Human enteroids establishment. Human small intestinal tissue was washed with ice-cold Dulbecco's Phosphate buffered saline without Ca2+ and Mg2+(DPBS), and secured on a silicone-coated glass Petri dish filled with ice-cold DPBS. The overlying mucosa from the submucosa and connective tissue was then removed. The mucosa was washed 3-4 times with ice-cold chelation buffer to remove villi and debris, and digested with freshly prepared 8 mM EDTA chelation buffer for 30 min on a horizontal orbital shaker, and cultured in human enteroids complete medium (AdDMEM/F12 medium composed 50% LWRN conditioned medium, HEPES, Glutamax, penicillin/streptomycin, B27, N-acetyl-L-cysteine, epidermal growth factor) mixed with Matrigel. The medium was replaced every other day. (See, e.g., Hill and Spence, Cell Mol Gastroenterol Hepatol 2017; 3:138-149).

Microinjection of human and mice enteroids. After treatment, each group of enteroids was checked before injection. Thin-wall glass capillaries and tips were prepared (See, e.g., Hill and Spence, Cell Mol Gastroenterol Hepatol 2017; 3:138-149). In the hood, the capillaries were filled with Fluorescein isothiocyanate-dextran (FD4) and then loaded onto the microinjector (BRI XenoWorks analog microinjector; Sutter Instrument Company). FD4 was used to aid in visualizing the injections. Each well medium was replaced after wash. The enteroids were imaged using a fluorescent stereomicroscope (SZX16; Olympus) at 1 magnification. Images were taken at the indicated time points post injection. The disruption of barrier integrity was determined by the loss of FD4 in the lumen of the enteroids.

Real-time quantitative polymerase chain reaction. qPCR and immunofluorescence image studies were used to determine if p40 was responsible for altering and/or improving mucosal permeability in the absence of immune modulation

### Example 2

### Metabolites of Lactobacillus rhamnosus GG (LGG) Prevent INF-γ-Induced Downregulation of Tight Junction Proteins in Mouse Enteroids

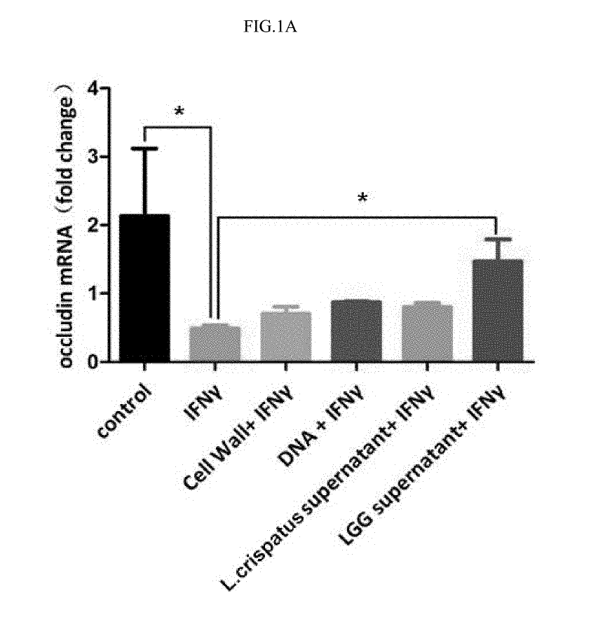

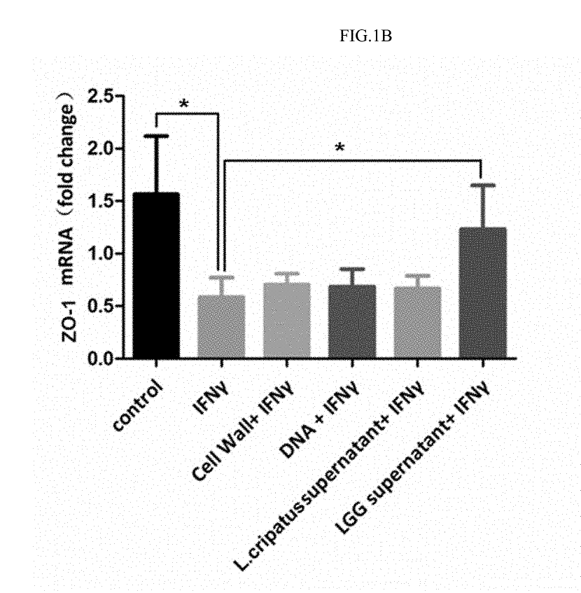

Mouse enteroids incubated with INF-γ resulted in a 80% and 67% downregulation of gene expression of occludin and ZO-1 (P<0.05), respectively (See FIGS. 1A and 1B). Pretreatment with LGG supernatant prevented these changes and normalized occludin and ZO-1 to control levels. In contrast, additions of LGG extracted DNA, boiled LGG Supernatant, LGG cell wall and cell-free L. cripatus supernatant did not significantly alter expression levels. * P<0.05 (See FIGS. 1A and 1B).

### Example 3

### Subcloning, Expression, Purification of Recombinant LGG Soluble Protein p40, and Immunodepletion of p40 from LGG Supernatant

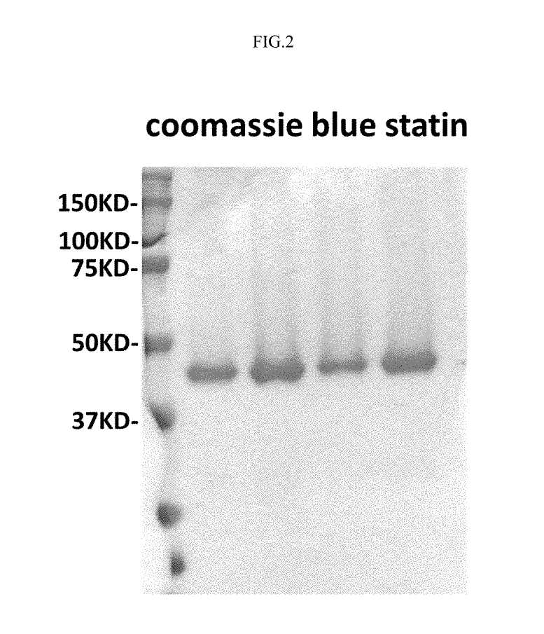

The coding sequence for p40 was PCR amplified from LGG genomic DNA. PCR fragments were cleaved and ligated into the pET28b+ expression vector. The recombinant plasmids were propagated in E. coli for expression of 41.5-kDa (p40). Cell suspensions were treated by centrifugation and applied to a nickel nitrilotriacetic acid (Ni-NTA) agarose column. Purified p40 was buffer exchanged to 10 mM Tris-HCl with a desalting column and then concentrated. Eluted proteins were separated by SDS-PAGE and stained with Colloidal Blue Staining in order to observe recombinant purified p40 protein (See FIG. 2).

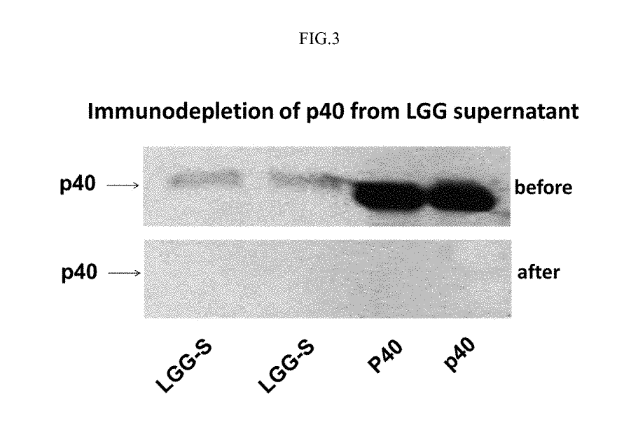

Polyclonal antibodies against recombinant p40 and LGG supernatant were generated and purified, and used in Western blot analysis (See FIG. 3). Antibody binding to recombinant p40 and p40 in LGG supernatant is shown in the upper panel of FIG. 3. Antibody binding to recombinant p40 and p40 in LGG supernatant after immunodepletion of p40 from LGG supernatant and recombinant p40 is shown in the lower panel of FIG. 3.

### Example 4

### P40 is Responsible for Improving Mucosal Permeability in the Absence of Immune Modulation and Apoptosis

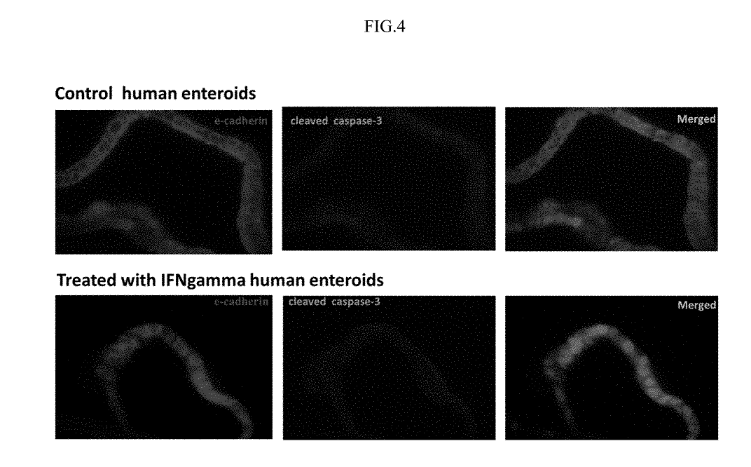

Human enteroids were treated with INFγ for 24 hr in the presence or absence of recombinant p40 or LGG supernatant. Immunofluorescence showed that cleaved caspase-3 (green fluorescence) were not expressed in IFNγ group indicating the absence of apoptosis (See FIG. 4).

### Example 5

### Immunodepletion of p40 from LGG Supernatant Abolished the Supernatant's Ability to Prevent Downregulation of ZO-1 and Occludin

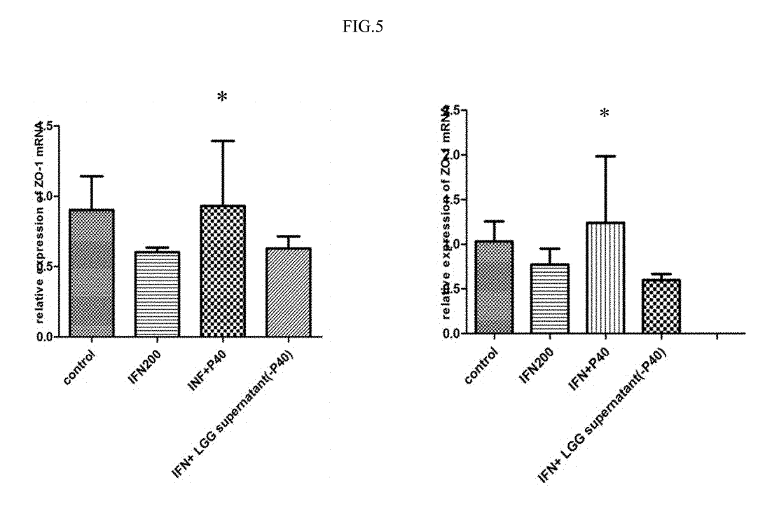

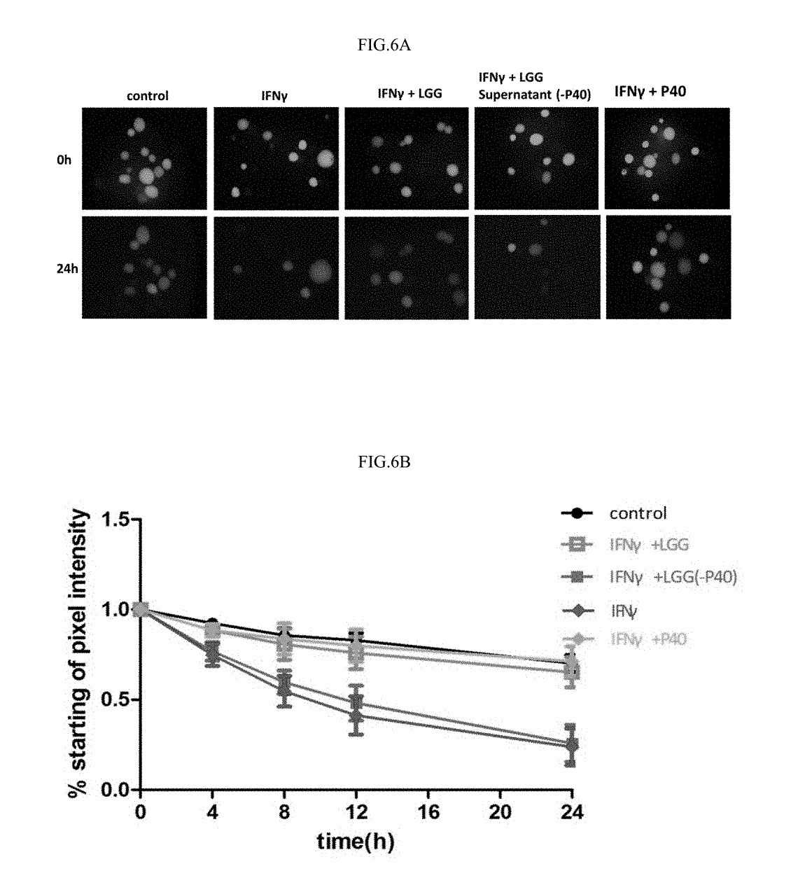

Using real time PCR, it was observed that incubation of human enteroids with INFγ for 24 hr caused a 30% and 20% downregulation of gene expression of occludin and ZO-1 (P<0.05) (See FIGS. 5, 6A and 6B). Downregulation was prevented by p40 (See FIG. 5). Immunodepletion of p40 from LGG supernatant abolished the ability of the supernatant to prevent downregulation of ZO-1 and occludin. * P<0.05 (FIG. 5).

FIG. 6 shows immunofluorescence staining of human enteroids with FD4. Under control conditions, the human enteroids retained 50% of FD4 at 24 hr (See FIGS. 6A and 6B). Treatment of the enteroids with INF gamma impaired permeability resulting in 20% retention of the dye. Administration of p40 prevented excessive leakage of dye evoked by INF gamma. * P<0.05 (See FIGS. 6A and 6B).

### Example 6

### Soluble p40 from LGG Supernatant Prevents Downregulation of ZO-1 and Occludin Gene Expression Induced by IBS-FSN

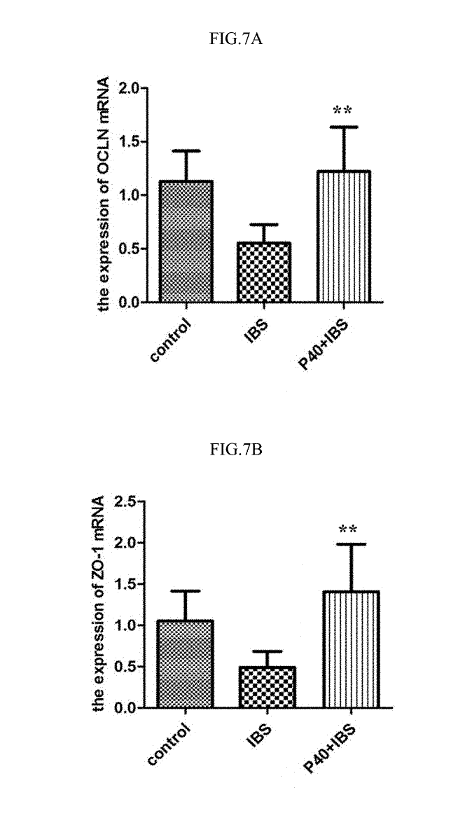

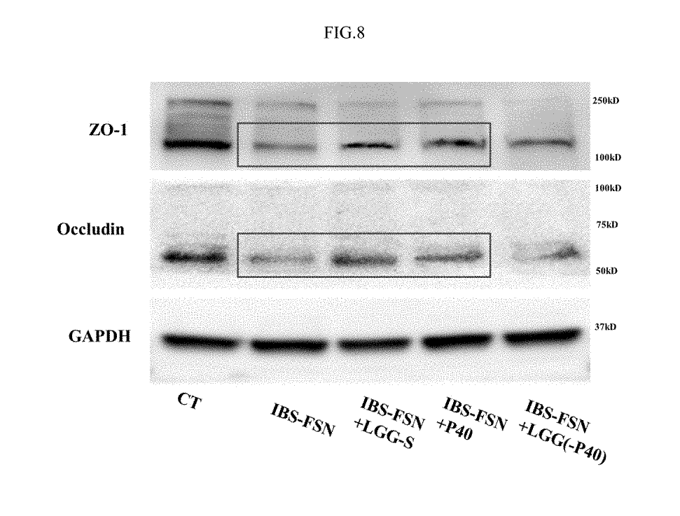

Incubation of human colonoids with Fecal supernatant from IBS-D patients induced epithelial barrier damage resulting in a 40% and 50% reduction in gene expression of occludin and ZO-1 (See FIG. 7). Treatment with p40 prevented the reduction and normalized the gene expression of ZO-1 and occludin (See FIG. 7).

### Example 7

### P40 Prevents IBS-FSN Induced Reduction of Mucosal Tight Junction

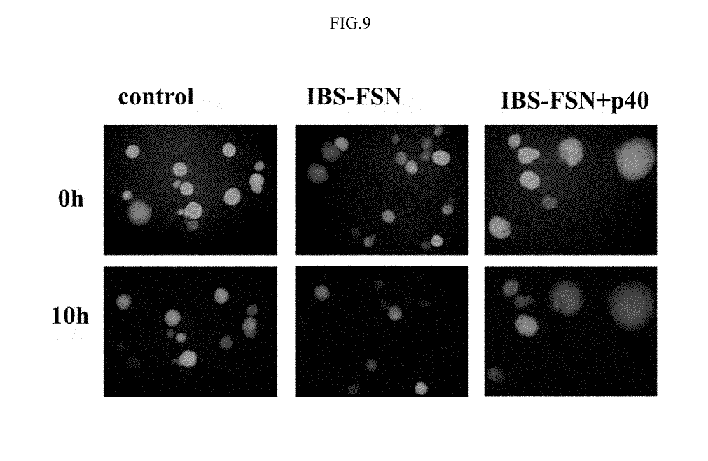

Administration of IBS-D patients fecal supernatant to human colonoids reduced protein expression of ZO-1 and Occludin (See FIG. 8). Pretreatment of the human colonoids with p40 prevented the reduction of ZO-1 and Occludin expression evoked by fecal supernatant of IBS-D patients. Removal of p40 by immunodepletion abolished the protective effects of LGG supernatant.

### Example 8

### P40 Prevents IBS-FSN Induced Disruption of Mucosal Permeability

Under control conditions, human colonoids retained 70% of FD4 over 10 h. Treatment of the colonoids with IBS-FSN impaired permeability resulting in 10% retention of the dye at 10 h (See FIG. 9). Administration of soluble protein p40 prevented leakage of dye evoked by IBS-FSN (See FIG. 9).

### Example 9

### In Vivo Studies Using P40 Produced by Lactobacillus rhamnosus GG (LGG) Enhanced Tight Junction Protein Expression and Prevented Epithelial Inflammation and the Development of Visceral Hypersensitivity

In order to confirm the in vitro organoid discoveries described in Examples 1-8, in vivo experiments were performed in rats. Intracolonic infusion of fecal supernatant from symptomatic Irritable Bowel Syndrome (IBS) patients in naïve rats has recently been shown to induce submucosal inflammation, intestinal barrier dysfunction and development of visceral hypersensitivity (see Zhou et al., J Clin Invest 2018: 128(1) 267-280). This was mediated by elevated fecal levels of lipopolysaccharide (LPS) due to gut dysbiosis. This rodent model of gut inflammation was used to examine the ability of P40 secreted by Lactobacillus rhamnosus GG (LGG) to ameliorate colonic inflammation and visceral hypersensitivity.

Materials and Methods.

Adult male Wistar rats (200-220 g) were divided into three groups (n=4-6 in each group). The placebo group received an intracolonic infusion of physiological buffer solution (PBS) (0.3 ml injected slowly for 1 minute) via a flexible plastic tube (18 gauge, 3 inches, Instech Laboratories) inserted into the distal colon 3 inches from the anus (JCI 2018). The second group received fecal supernatant (0.3 ml) given in a similar manner and the third group was pretreated with P40 protein (30 μg in 0.3 ml of PBS) given intracolonically three times, each 1 hr apart, prior to infusion of fecal supernatant. Pain behavior studies (VMR to graded colorectal distension) were performed 5 hr after infusion of the fecal supernatant. Subsequently, rats were sacrificed and left colon tissues were collected for TEER (in vivo gut permeability measurement) and qPCR studies for cytokines and junction proteins (Z01 and occludin) measurement.

Results.

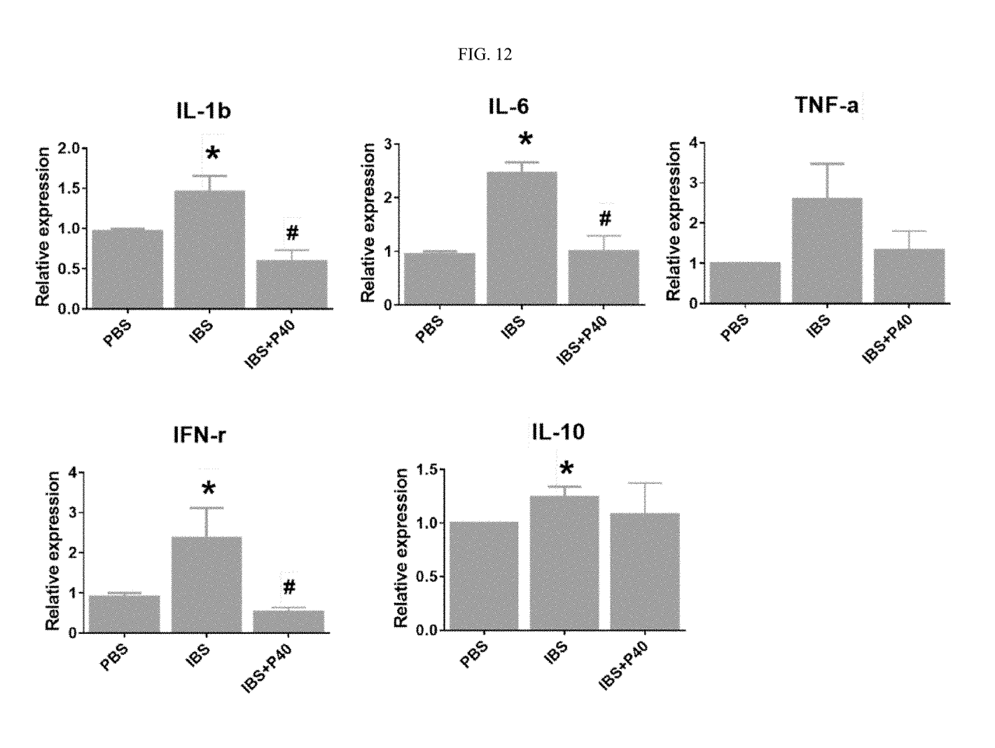

Intracolonic administration of fecal supernatant from IBS patients increased gene expression of Il-Iβ, IFNγ and IL6 in the left colon compared with the administration of PBS (student's T test n=4-6, P<0.05) (See FIG. 12).

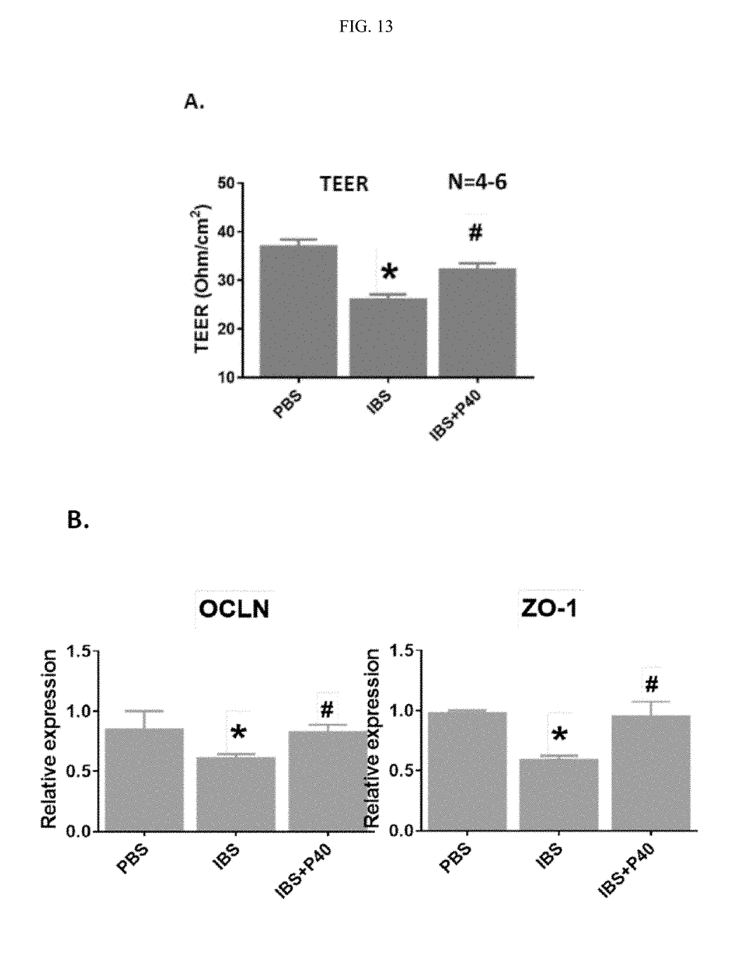

Colonic barrier function was evaluated by measuring the transepithelial electrical resistance (TEER) of ex vivo colonic tissues. TEER reflects paracellular space and is a sensitive measure of barrier integrity. Colonic mucosa resistance was reduced in rats following intracolonic infusion of fecal supernatant from IBS patients compared to those receiving PBS infusion (n=4-6, P<0.05) (See FIG. 13A). A reduced TEER was accompanied by reduced expression of epithelial tight junction proteins zonula occludens-1 (Z01) and occludin (OCLN) by 39% and 24% respectively (See FIG. 13B).

Intracolonic infusion of P40 protein prevented IBS fecal supernatant-provoked cytokine gene expression. (See FIG. 12). In addition, P40 infusion normalized the increase in TEER induced by IBS fecal supernatant (See FIG. 13A) and prevented a reduction on tight junction proteins Z01 and occludin in the left colon (See FIG. 13B).

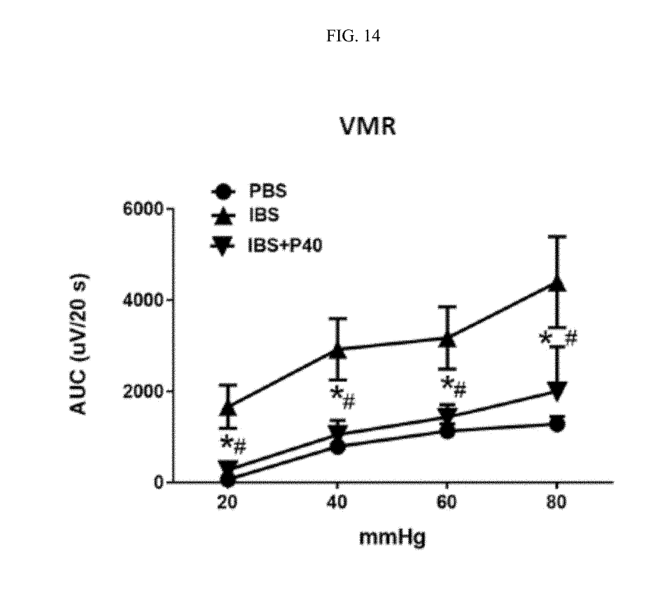

In separate experiments conducted during development of embodiments of the invention, pain behavior testing showed VMR to 20, 40, 60 and 80 MmHg colorectal distension significantly increased in rats treated with intracolonic infusion of IBS fecal supernatant compared with controls (n=4-6, P<0.05) (See FIG. 14). However, pretreatment of intracolonic infusion of P40 protein normalized the VMR to colorectal distension evoked by IBS fecal supernatant (See FIG. 14).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

What is claimed is:

1. A composition comprising bacteria genetically engineered to express recombinant Lactobacillus p40 protein.

2. The composition of claim 1, wherein the genetically engineered bacteria is Lactobacillus.

3. The composition of claim 2, wherein the Lactobacillus is Lactobacillus rhamnosus GG (LGG).

4. The composition of claim 2, further comprising one or more non-Lactobacillus strains of bacteria.

5. A cell lysate prepared from a composition of claim 1.

6. A composition comprising bacterial cells and recombinant Lactobacillus p40 protein.

7. The composition of claim 6, wherein the recombinant p40 protein is isolated and/or purified.

8. A method of treating a gastrointestinal disease or disorder in a subject comprising administering to the subject a therapeutically effective amount of a composition comprising recombinant Lactobacillus p40 protein.

9. The method of claim 8, wherein the composition comprising recombinant Lactobacillus p40 protein comprises bacteria genetically engineered to express recombinant Lactobacillus p40 protein.

10. The method of claim 9, wherein the genetically engineered bacteria is Lactobacillus.

11. The method of claim 10, wherein the Lactobacillus is Lactobacillus rhamnosus GG (LGG).

12. The method of claim 10, wherein the composition further comprising one or more non-Lactobacillus strains of bacteria.

13. The method of claim 8, wherein the composition comprising recombinant Lactobacillus p40 protein comprises a cell lysate from bacteria genetically engineered to express recombinant Lactobacillus p40 protein.

14. The method of claim 8, wherein the gastrointestinal disorder or disease is irritable bowel syndrome (IBS).

15. The method of claim 8, wherein the gastrointestinal disorder or disease is inflammatory bowel disease (IBD).

16. The method of claim 8, wherein the administering alters intestinal mucosal epithelial barrier function in the subject.

17. The method of claim 16, wherein altering intestinal mucosal epithelial barrier function comprises improving mucosal epithelial barrier function.

18. The method of claim 16, wherein altering intestinal mucosal epithelial barrier function comprises reducing intestinal mucosal tight junction damage.
